(19) United States
(12) Patent Application Publication
(10) Pub. No.: US 2026/0347472 A1
(43) Pub. Date: Nov. 5, 2026

(54) RECOMBINANT ANTIBODY AND USE THEREOF

(71) Applicant: HEFEI TG IMMUNOPHARMA CO., LTD., Hefei (CN)

(72) Inventors: Guoshuai Cao, Hefei (CN); Ying Cheng, Hefei (CN); Yangyang Li, Hefei (CN); Yuwei Wu, Hefei (CN)

(21) Appl. No.: 19/703,385

(22) Filed: Jun. 10, 2026

Related U.S. Application Data
(63) Continuation of application No. PCT/CN2024/139038, filed on Dec. 13, 2024.

(30) Foreign Application Priority Data
Dec. 13, 2023 (CN) 202311720119.1

(51) Int. Cl. C07K 16/28; A61K 47/68; A61K 51/10; A61P 35/00; C07K 16/40; G01N 33/564; G01N 33/575

(52) U.S. Cl. CPC C07K 16/2809; A61K 47/6811; A61K 51/1042; A61P 35/00; C07K 16/40; G01N 33/564; G01N 33/5758; C07K 2317/31; C07K 2317/622; G01N 2333/7051; G01N 2333/96433

(57) ABSTRACT

The present disclosure relates to the field of biomedicine, and specifically, to a recombinant antibody and use thereof. A CD3 and FAP bispecific antibody binds weakly to T cells, binds strongly to FAP-positive cells, and has high pro-killing activity, effectively promoting killing of the FAP-positive cells by PBMCs and demonstrating satisfactory anticancer activity. In addition, the bispecific antibody has reduced capability in promoting pro-inflammatory cytokine secretion and T cell activation, thereby possessing higher safety.

### CROSS-REFERENCE TO RELATED APPLICATIONS

This application is a continuation of International Application No. PCT/CN2024/139038, filed on Dec. 13, 2024, which claims priority to claims priority to Chinese Patent Application No. 202311720119.1, titled “RECOMBINANT ANTIBODY AND USE THEREOF”, and filed with China National Intellectual Property Administration on Dec. 13, 2023, the entire contents of which are incorporated herein by reference.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence Listing.xml”, was created on Jun. 4, 2026, edited on Jun. 9, 2026, and is 40,876 bytes measured in Microsoft Windows operating system.

### FIELD

The present disclosure relates to the field of biomedicine, and specifically, to a recombinant antibody and use thereof.

### BACKGROUND

Cancer is a major disease affecting human survival and development. According to the latest data, the number of new cancer cases is up to about 19 million every year, and the number of cancer-caused death cases is up to about 10 million per year worldwide. The incidence and mortality of cancers exhibit an increasing trend. Except for surgical resection, the conventional cancer treatment methods such as chemotherapy and radiotherapy have disadvantages of significant side effects and easy recurrence. In recent years, immunotherapy, including tumor-targeted antibodies, immune checkpoint antibodies, and bispecific antibodies, has become a new focus and a promising approach against cancers. The immunotherapy represented by PD-1/L1 exhibits huge potential. However, even the currently PD-1/L1 therapy with the broadest approved indications has an overall response rate of only 30%, and more patients cannot benefit from the therapy. One of the main reasons is that the immune checkpoint therapy is ineffective to “cold tumors”. T cells recognize neo-antigens, i.e., antigens generated by tumor gene mutations, through T cell receptors (TCR) on the surfaces. However, some tumors with low frequency of gene mutation and few types of neo-antigens are referred to as “cold tumors”. Current immune checkpoint therapy such as the PD-1/L1 therapy aims to fight cancer by restoring the intrinsic functions of T cells. However, in “cold tumors”, T cells cannot effectively recognize the “cold tumors”. Therefore, the immune checkpoint therapy is ineffective to the “cold tumors”.

CD3-based bispecific antibodies (hereinafter referred to as “CD3 bispecific antibodies”) can promote T cell activation and kill tumors by recruiting T cells to reach the tumor site, and bridging T cells with tumors. Such bispecific antibodies do not require neo-antigens and can guide T cells to kill the “cold tumors”. The CD3 bispecific antibodies can trigger strong activation signals upon binding to T cells and tumor cells, and they can thus also “ignore” inhibitory signals of immune checkpoint molecules to some extent. However, the CD3 bispecific antibodies can also promote generation of a large number of pro-inflammatory cytokines, such as TNFα and IL-6, which triggers a strong cytokine storm and excessive immune response, causing damage to the body and potentially endangering life in severe cases. Therefore, the CD3-based bispecific antibodies have promising clinical applications, but their safety needs to be further improved.

One approach to solve the safety issues of the CD3 bispecific antibodies is to increase their affinity for binding to tumor targets while reducing their affinity for CD3, thereby enabling the CD3 bispecific antibody drugs to be distributed more extensively to the tumor site, increasing local drug concentration at the tumor site, reducing the peripheral drug concentration, lowering off-target toxicity, reducing a production level of pro-inflammatory cytokines, and lowering on-target toxicity. Therefore, there is an urgent need in clinical practice to develop CD3 bispecific antibodies with better safety and higher clinical application value.

Fibroblast activation protein (FAP), is a type II transmembrane protein belonging to the serine protease family. It possesses serine proteolytic enzyme activity, participates in extracellular matrix degradation, and is associated with tumor growth, metastasis, etc. FAP is not expressed in normal tissues, but is highly upregulated in many cancers, specifically expressed on the surface of tumor fibroblasts. It can also be hydrolyzed by other proteolytic enzymes into a soluble form existing in tissue fluid and plasma, promoting tumor cell migration and invasion through extracellular matrix hydrolysis. Due to the characteristic of tumor tissue-specific expression of FAP, directly targeting and killing tumor fibroblasts expressing FAP to destroy the tumor microenvironment, or using FAP antibodies to target tumor tissues to deliver drugs, has become a hotspot in immunotherapy drug development.

In summary, FAP is a promising therapeutic target, and developing a CD3×FAP bispecific antibody having high safety and strong specificity is of great value.

### SUMMARY

The present disclosure aims to solve at least one of the technical problems in the related art to some extent.

In a first aspect of the present disclosure, a recombinant antibody is provided. The recombinant antibody at least includes: a first binding region, in which the first binding region includes an antibody or antigen-binding fragment that specifically recognizes CD3; and a second binding region, in which the second binding region includes an antibody or antigen-binding fragment that specifically recognizes FAP. The antibody or antigen-binding fragment that specifically recognizes the CD3 in the first binding region includes an scFV region and a first Fc region. The antibody or antigen-binding fragment that specifically recognizes the FAP in the second binding region includes a Fab region and a second Fc region.

To improve the safety of the CD3 and FAP recombinant antibody, the present disclosure has obtained a CD3 and FAP bispecific antibody having a new configuration through extensive screening and experimental verification. Further experimental results demonstrate that the CD3 and FAP bispecific antibody of the present disclosure binds weakly to T cells, but binds strongly to FAP-positive cells, and has high pro-killing activity, which can effectively promote killing of the FAP-positive cells by peripheral blood mononuclear cells (PBMCs) and demonstrating satisfactory anticancer activity. In addition, the CD3 and FAP bispecific antibody has reduced capability in promoting pro-inflammatory cytokine secretion and T cell activation, thereby possessing higher safety. Thus, the CD3 and FAP bispecific antibody has satisfactory clinical application and drug development value.

According to an embodiment of the present disclosure, the above recombinant antibody can further include at least one of the following additional technical features.

According to a specific embodiment of the present disclosure, in the first binding region, a C-terminus of the scFV region is linked to an N-terminus of the first Fc region.

According to a specific embodiment of the present disclosure, a C-terminus of a heavy chain variable region of the scFV region is linked to an N-terminus of a light chain variable region via a linker peptide 1, and a C-terminus of the light chain variable region is linked to the N-terminus of the first Fc region via a linker peptide 2.

According to a specific embodiment of the present disclosure, the antibody or antigen-binding fragment that specifically recognizes the FAP includes: light chain variable region CDR1, CDR2, CDR3 sequences as set forth in SEQ ID NO: 12, SEQ ID NO: 13, and SEQ ID NO: 14, respectively, and/or heavy chain variable region CDR1, CDR2, CDR3 sequences as set forth in SEQ ID NO:9, SEQ ID NO: 10, and SEQ ID NO: 11, respectively.

According to a specific embodiment of the present disclosure, the antibody or antigen-binding fragment that specifically recognizes the FAP has a light chain variable region as set forth in SEQ ID NO: 31 or an amino acid sequence having at least 80% identity to a sequence set forth in SEQ ID NO: 31, and a heavy chain variable region as set forth in SEQ ID NO: 32 or an amino acid sequence having at least 80% identity to a sequence set forth in SEQ ID NO: 32.

According to a specific embodiment of the present disclosure, the antibody or antigen-binding fragment that specifically recognizes the FAP has a light chain as set forth in SEQ ID NO: 20 or an amino acid sequence having at least 80% identity to a sequence set forth in SEQ ID NO: 20, and a heavy chain as set forth in SEQ ID NO: 19 or an amino acid sequence having at least 80% identity to a sequence set forth in SEQ ID NO: 19.

According to a specific embodiment of the present disclosure, the antibody or antigen-binding fragment that specifically recognizes the CD3 includes: light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 3, respectively; light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 6, respectively; or light chain variable region CDR1, CDR2, CDR3 sequences as set forth in SEQ ID NO: 4, GTN, and SEQ ID NO:8, respectively, and heavy chain variable region CDR1, CDR2, CDR3 sequences as set forth in SEQ ID NO: 1, SEQ ID NO: 2, and SEQ ID NO: 7, respectively.

According to a specific embodiment of the present disclosure, the antibody or antigen-binding fragment that specifically recognizes the CD3 includes any one of: (a) a light chain variable region as set forth in SEQ ID NO: 26 and a heavy chain variable region as set forth in SEQ ID NO: 27; (b) a light chain variable region as set forth in SEQ ID NO: 26 and a heavy chain variable region as set forth in SEQ ID NO: 28; (c) a light chain variable region as set forth in SEQ ID NO: 29 and a heavy chain variable region as set forth in SEQ ID NO: 30; or amino acid sequences having at least 80% sequence identity to any one of (a), (b), and (c).

According to a specific embodiment of the present disclosure, the first Fc region is linked to the second Fc region via a knob-into-hole structure.

According to a specific embodiment of the present disclosure, at least part of each of the first Fc region and/or the second Fc region is from at least one of a murine antibody, a primate-derived antibody, or a mutant thereof.

According to a specific embodiment of the present disclosure, at least part of each of the first Fc region and/or the second Fc region is from human IgG1 or a mutant thereof.

According to a specific embodiment of the present disclosure, the first Fc region has an amino acid sequence as set forth in SEQ ID NO: 33, and/or the second Fc region has an amino acid sequence as set forth in SEQ ID NO: 34.

According to a specific embodiment of the present disclosure, the linker peptide 1 and the linker peptide 2 have ach independently an amino acid sequence (GGGGS)n, where n is an integer greater than or equal to 1, and preferably n is 1, 2, 3, 4, 5, 6, 7, 8, 9, or 10.

According to a specific embodiment of the present disclosure, the linker peptide 1 has an amino acid sequence as set forth in SEQ ID NO: 35.

According to a specific embodiment of the present disclosure, the linker peptide 2 has an amino acid sequence as set forth in SEQ ID NO: 36.

According to a specific embodiment of the present disclosure, the first binding region includes an amino acid sequence as set forth in any one of SEQ ID NO: 15, SEQ ID NO: 16, or SEQ ID NO: 17, or an amino acid sequence having at least 80% identity to SEQ ID NO: 15, SEQ ID NO: 16, or SEQ ID NO: 17.

According to a specific embodiment of the present disclosure, the recombinant antibody includes any one of: (d) amino acid sequences as set forth in SEQ ID NO: 15, SEQ ID NO: 19, and SEQ ID NO: 20; (e) amino acid sequences as set forth in SEQ ID NO: 16, SEQ ID NO: 19, and SEQ ID NO: 20; (f) amino acid sequences as set forth in SEQ ID NO: 17, SEQ ID NO: 19, and SEQ ID NO: 20; or amino acid sequences having at least 80% sequence identity to any one of (d), (e), and (f).

According to a specific embodiment of the present disclosure, the recombinant antibody further includes a third binding region. The third binding region includes the antibody or antigen-binding fragment that specifically recognizes the FAP.

According to a specific embodiment of the present disclosure, the antibody or antigen-binding fragment that specifically recognizes the FAP in the third binding region includes a Fab region, a heavy chain CH1 region of the Fab region being linked to the scFV region of the antibody or antigen-binding fragment that specifically recognizes the CD3 in the first binding region.

According to a specific embodiment of the present disclosure, a C-terminus of the heavy chain CH1 region of the Fab region is linked to a heavy chain variable region of the scFV region of the antibody or antigen-binding fragment that specifically recognizes the CD3 in the first binding region.

According to a specific embodiment of the present disclosure, an amino acid sequence of the Fab region of the antibody or antigen-binding fragment that specifically recognizes the FAP in the third binding region and an amino acid sequence of the Fab region of the antibody or antigen-binding fragment that specifically recognizes the FAP in the second binding region are identical or different.

In a second aspect of the present disclosure, an isolated polynucleotide is provided. The polynucleotide encodes the recombinant antibody described in the first aspect.

According to an embodiment of the present disclosure, the above polynucleotide can further include at least one of the following additional technical features.

According to an embodiment of the present disclosure, the antibody or antigen-binding fragment encoded by the isolated polynucleotide is capable of specifically binding to FAP and/or CD3.

According to an embodiment of the present disclosure, a nucleic acid molecule of the polynucleotide is DNA and/or RNA.

It should be noted that, for the nucleic acids mentioned in the description and claims of the present disclosure, those skilled in the art should understand that any one or two of complementary double strands are actually included. For convenience, although in most cases only one strand is given in the present description and the claims, the other strand complementary to the one strand is also disclosed actually. In addition, nucleotide sequences in the present disclosure include a DNA form or an RNA form. When one form is disclosed, it means that the other form is also disclosed.

In a third aspect of the present disclosure, an expression vector is provided. The expression vector carries the polynucleotide described in the second aspect.

According to an embodiment of the present disclosure, the expression vector may include an optional control sequence. The control sequence is operably linked with the nucleic acid molecule. The control sequence refers to one or more control sequences that can direct the expression of said nucleic acid molecule in the host. The expression vector provided in an embodiment of the present disclosure can efficiently and highly express the antibody or antigen-binding fragment thereof in suitable host cells.

In a fourth aspect of the present disclosure, a recombinant cell is provided. The recombinant cell carries the polynucleotide described in the second aspect or the expression vector described in the third aspect, or is capable of expressing the recombinant antibody described in the first aspect.

According to an embodiment of the present disclosure, the above recombinant cell can further include at least one of the following additional technical features.

According to an embodiment of the present disclosure, the recombinant cell is obtained by introducing the expression vector described in the fourth aspect into a host cell.

According to an embodiment of the present disclosure, the recombinant cell is a eukaryotic cell.

According to an embodiment of the present disclosure, the recombinant cell is a mammalian cell.

It should be noted that, the recombinant cell according to the present disclosure is not particularly limited, and it may be a prokaryotic cell, a eukaryotic cell, or a bacteriophage. The prokaryotic cell may be Escherichia coli, Bacillus subtilis, Streptomyces, or Proteus mirabilis. The eukaryotic cell includes: fungi such as Pichia pastoris, Saccharomyces cerevisiae, Schizosaccharomyces, and Trichoderma, insect cells such as grass armyworms; plant cells such as tobacco; and mammalian cells such as BHK cells, CHO cells, COS cells, and myeloma cells. In some embodiments, the recombinant cells according to the present disclosure are preferably the mammalian cells, including BHK cells, CHO cells, NSO cells, or COS cells and do not include animal germ cells, fertilized egg or embryonic stem cells.

According to an embodiment of the present disclosure, when the cell is the eukaryotic cell, such as the mammalian cell, the expression efficiency of the recombinant antibody is high.

In a fifth aspect of the present disclosure, a composition is provided. The composition includes at least one of the recombinant antibody described in the first aspect, the polynucleotide described in the second aspect, the expression vector described in the third aspect, or the recombinant cell described in the fourth aspect.

According to an embodiment of the present disclosure, the recombinant antibody can effectively promote the killing of the FAP-positive cells by the PBMCs and have the anticancer activity. In addition, the recombinant antibody can produce fewer pro-inflammatory cytokines, has higher safety, and possesses satisfactory clinical application value and drug development value. Thus, the obtained drug can be further used for preventing and/or treating a CD3-mediated related disease and/or an FAP-mediated related disease.

According to an embodiment of the present disclosure, the recombinant antibody can effectively bind to FAP protein and CD3 and effectively inhibit the proliferation of tumor cells. As a result, the composition including the above-mentioned substances can also effectively bind to FAP and CD3 proteins and thus have good effects in preventing and/or treating the FAP-mediated disease. The type of the composition is not particularly limited. The composition may be a food composition or a pharmaceutical composition.

The composition according to the present disclosure can be combined with each other, or it can be administered in combination with one or more other therapeutic compounds, for example, in combination with a chemotherapeutic agent. Therefore, the composition may further include a chemotherapeutic agent. The antibody or the antigen-binding fragment thereof or the immunoconjugate according to the present disclosure may be in combination with a second therapeutic agent. Exemplary agents of the second therapeutic agent include, but not limited to: other reagents that inhibit FAP activity, including other antibodies or antigen-binding fragments thereof, peptide inhibitors, small molecule antagonists, etc.; and/or agents interfering with upstream or downstream signal transduction of FAP.

It should be noted that, the composition includes combinations separated temporally and/or spatially, as long as they are capable of acting together to achieve the objectives of the present disclosure. For example, the components contained in the composition may be applied to a subject as a whole or separately. When the components contained in the composition are separately administered to the subject, the respective components may be simultaneously or sequentially administered to the subject.

In a sixth aspect of the present disclosure, a method for preparing the recombinant antibody described in the first aspect is provided. The method includes: culturing the recombinant cell described in the fourth aspect, and isolating the antibody or antigen-binding fragment of the recombinant cell.

In a seventh aspect of the present disclosure, a medicament is provided. The medicament includes at least one of the recombinant antibody described in the first aspect, the polynucleotide described in the second aspect, the expression vector described in the third aspect, the recombinant cell described in the fourth aspect, or the composition described in the fifth aspect.

In an eighth aspect of the present disclosure, a kit is provided. The kit includes at least one of the recombinant antibody described in the first aspect, the polynucleotide described in the second aspect, the expression vector described in the third aspect, or the recombinant cell described in the fourth aspect.

In a ninth aspect of the present disclosure, use of the recombinant antibody described in the first aspect, the polynucleotide described in the second aspect, the expression vector described in the third aspect, the recombinant cell described in the fourth aspect, or the composition described in the fifth aspect in the manufacture of a medicament is provided. The medicament is used for preventing and/or treating an FAP-mediated related disease.

According to an embodiment of the present disclosure, the above use can further include at least one of the following additional technical features.

According to an embodiment of the present disclosure, the FAP-mediated related disease includes a tumor, tumor-associated fibroblasts of the tumor being FAP-positive.

According to an embodiment of the present disclosure, a disease caused by the tumor includes lung cancer, liver cancer, ovarian cancer, cervical cancer, skin cancer, bladder cancer, colon cancer, breast cancer, glioma, kidney cancer, gastric cancer, melanoma, esophageal cancer, oral squamous cell carcinoma, or head-and-neck cancer.

In a tenth aspect of the present disclosure, a kit for detecting FAP and/or CD3 in a sample is provided. The kit includes at least one of the recombinant antibody described in the first aspect, the polynucleotide described in the second aspect, the expression vector described in the third aspect, or the recombinant cell described in the fourth aspect.

According to an embodiment of the present disclosure, the kit including the antibody or antigen-binding fragment thereof can effectively perform qualitative or quantitative detection of the FAP protein and/or CD3. The kit provided by the present disclosure, may be used, for instance, in applications such as Western blotting, immunoprecipitation, or other assays that utilize the specific binding of human FAP and the antibody. Such a kit may include any one or more of an antagonist, an anti-FAP antibody, an anti-CD3 antibody, or a drug reference material; a protein purification column; an immunoglobulin affinity purification buffering agent; an assay diluent for cells; an instruction or literature. The anti-FAP antibody and/or CD3 may be used for different types of diagnostic tests, for example, an in vitro or in vivo detection of various diseases or presence of a medicament, a toxin, or other proteins. For example, serum or blood samples from subjects can be tested for the diagnosis of relevant diseases, or scientific research can be performed by detecting FAP protein in the sample to be tested. The related diseases may include an FAP related disease, such as cancer. Certainly, the antibody or antigen-binding fragment thereof provided in the present disclosure may also be used for radioimmunoassay and radioimmunotherapy of the above diseases. With respect to the above application scenarios, the binding molecule is also applicable, which is not described in detail herein.

In an eleventh aspect of the present disclosure, use of the recombinant antibody described in the first aspect, the polynucleotide described in the second aspect, the expression vector described in the third aspect, or the recombinant cell described in the fourth aspect in the preparation of a kit is provided. The kit is used for detecting FAP and/or CD3.

In a twelfth aspect of the present disclosure, an immunoconjugate is provided. The immunoconjugate includes a therapeutic agent and the recombinant antibody described in the first aspect conjugated to the therapeutic agent.

According to an embodiment of the present disclosure, the therapeutic agent includes any one of a polypeptide, a radionuclide, and a small molecule.

In a thirteenth aspect of the present disclosure, use of the recombinant antibody described in the first aspect, the polynucleotide described in the second aspect, the expression vector described in the third aspect, the recombinant cell described in the fourth aspect, the composition described in the fifth aspect, or the medicament described in the seventh aspect in preventing and/or treating a CD3-mediated related disease and/or an FAP-mediated related disease is provided.

In a fourteenth aspect of the present disclosure, the recombinant antibody described in the first aspect, the polynucleotide described in the second aspect, the expression vector described in the third aspect, the recombinant cell described in the fourth aspect, the composition described in the fifth aspect, or the medicament described in the seventh aspect, for use in preventing and/or treating a CD3-mediated related disease and/or an FAP-mediated related disease is provided.

In a fifteenth aspect of the present disclosure, use of the recombinant antibody described in the first aspect, the polynucleotide described in the second aspect, or the kit described in the tenth aspect in diagnosing whether a subject is suffering from a CD3-mediated related disease and/or an FAP-mediated related disease is provided.

In a sixteenth aspect of the present disclosure, a method for preventing and/or treating a CD3-mediated related disease and/or an FAP-mediated related disease is provided. The method includes: administering to a subject a pharmaceutically acceptable amount of the recombinant antibody described in the first aspect, the polynucleotide described in the second aspect, the expression vector described in the third aspect, the recombinant cell described in the fourth aspect, the composition described in the fifth aspect, or the medicament described in the seventh aspect.

In a seventeenth aspect of the present disclosure, a method for diagnosing whether a subject is suffering from a CD3-mediated related disease and/or an FAP-mediated related disease is provided. According to an embodiment of the present disclosure, the method includes: detecting a to-be-tested sample from the subject by using the recombinant antibody described in the first aspect, the polynucleotide described in the second aspect, or the kit described in the tenth aspect.

In an eighteenth aspect of the present disclosure, use of the recombinant antibody described in the first aspect, the polynucleotide described in the second aspect, or the kit described in the tenth aspect in detecting CD3 and/or FAP in a to-be-tested sample is provided.

In a nineteenth aspect of the present disclosure, a method for detecting CD3 and/or FAP is provided. According to an embodiment of the present disclosure, the method includes: detecting a to-be-tested sample using the recombinant antibody described in the first aspect, the polynucleotide described in the second aspect, or the kit described in the tenth aspect.

Additional aspects and advantages of the present disclosure will be provided at least in part in the following description, or will become apparent in part from the following description, or can be learned from the practice of the present disclosure.

### BRIEF DESCRIPTION OF THE DRAWINGS

The above and other aspects and advantages of embodiments of the present disclosure will become apparent and more readily appreciated from the following descriptions made with reference to the accompanying drawings.

FIG. 1 is a schematic diagram of a CD3×FAP bispecific antibody mode in Example 1 of the present disclosure.

FIG. 2 is a graph showing the flow cytometry results of binding of antibodies Cross3×AHF21339 1:1, Cross3×AHF21339 2:1, UCHT1×AHF21339 1:1, and UCHT1×AHF21339 2:1 to CD8 T cells in Example 2 of the present disclosure, in which A in FIG. 2 represents a binding percentage of antibody to CD8 T cells, and B in FIG. 2 represents a mean fluorescence intensity.

FIG. 3 is a graph showing the flow cytometry results of binding of antibodies Cross3×AHF21339 1:1, Cross313×AHF21339, Cross316×AHF21339, and Cross325×AHF21339 to CD8 T cells in Example 2 of the present disclosure, in which A in FIG. 3 represents a binding percentage of antibody to CD8 T cells, and B in FIG. 3 represents a mean fluorescence intensity.

FIG. 4 is a graph showing the flow cytometry results of binding of antibodies Cross3×AHF21339 1:1, Cross3×AHF21339 2:1, UCHT1×AHF21339 1:1, and UCHT1×AHF21339 2:1 to A-375-FAP cells in Example 2 of the present disclosure, in which A in FIG. 4 represents a binding percentage of antibody to A-375-FAP cells, and B in FIG. 4 represents a mean fluorescence intensity.

FIG. 5 is a graph showing the flow cytometry results of binding of antibodies Cross3×AHF21339 1:1, Cross313×AHF21339, Cross316×AHF21339, and Cross325×AHF21339 to A-375-FAP cells in Example 2 of the present disclosure, in which A in FIG. 5 represents a binding percentage of antibody to A-375-FAP cells, and B in FIG. 5 represents a mean fluorescence intensity.

FIG. 6 a graph showing the results of spontaneous activation and cytokine secretion of PBMCs promoted by Cross3×AHF21339 1:1 and UCHT1×AHF21339 1:1 antibodies in the absence of target protein (FAP) in Example 3 of the present disclosure, in which A in FIG. 6 represents a secretion result of IL-2, B in FIG. 6 represents a secretion result of IFN-γ, C in FIG. 6 represents a secretion result of IL-6, and D in FIG. 6 represents a secretion result of TNFα.

FIG. 7 a graph showing the results of spontaneous activation and IL-6 secretion of spontaneous activation of PBMCs promoted by Cross3×AHF21339 1:1, Cross313×AHF21339, Cross316×AHF21339, and Cross325×AHF21339 antibodies in Example 3 of the present disclosure.

FIG. 8 a graph showing the results of spontaneous activation and cytokine secretion of PBMCs promoted by Cross3×AHF21339 1:1, Cross3×AHF21339 2:1, UCHT1×AHF21339 1:1, and UCHT1×AHF21339 2:1 in the presence of target protein (FAP) in Example 3 of the present disclosure, in which A in FIG. 8 represents a secretion result of IL-2, and B in FIG. 8 represents a secretion result of IFN-γ.

FIG. 9 a graph showing the results of IL-2 secretion promoted by Cross3×AHF21339 1:1, Cross313×AHF21339, Cross316×AHF21339, and Cross325×AHF21339 antibodies in co-incubation of PBMCs with A375-FAP in Example 3 of the present disclosure.

FIG. 10 a graph showing the results of IFN-γ secretion promoted by Cross3×AHF21339 1:1, Cross313×AHF21339, Cross316×AHF21339, and Cross325×AHF21339 antibodies in co-incubation of PBMCs with A375-FAP in Example 3 of the present disclosure.

FIG. 11 a graph showing the results of TNFα secretion promoted by Cross3×AHF21339 1:1, Cross313×AHF21339, Cross316×AHF21339, and Cross325×AHF21339 antibodies in co-incubation of PBMCs with A375-FAP in Example 3 of the present disclosure.

FIG. 12 a graph showing the results of IL-6 secretion promoted by Cross3×AHF21339 1:1, Cross313×AHF21339, Cross316×AHF21339, and Cross325×AHF21339 antibodies in co-incubation of PBMCs with A375-FAP in Example 3 of the present disclosure.

FIG. 13 is a graph showing the flow cytometry results of T cell proliferation promoted by Cross3×AHF21339 1:1, Cross3×AHF21339 2:1, UCHT1×AHF21339 1:1, and UCHT1×AHF21339 2:1 antibodies in Example 4 of the present disclosure, in which A in FIG. 13 represents an activation status of CD4 T cells by the antibodies, and B in FIG. 13 represents an activation status of CD8 T cells by the antibodies.

FIG. 14 is a graph showing that Cross3×AHF21339 1:1, Cross313×AHF21339, Cross316×AHF21339, and Cross325×AHF21339 antibodies can all promote CD4 T cell proliferation in Example 4 of the present disclosure.

FIG. 15 is a graph showing that Cross3×AHF21339 1:1, Cross313×AHF21339, Cross316×AHF21339, and Cross325×AHF21339 antibodies can all promote CD8 T cell proliferation in Example 4 of the present disclosure.

FIG. 16 a graph showing the results of PBMC-mediated killing of A-375-FAP melanoma cells promoted by antibodies Cross3×AHF21339 1:1, Cross3×AHF21339 2:1, UCHT1×AHF21339 1:1, and UCHT1×AHF21339 2:1 in Example 5 of the present disclosure.

FIG. 17 is a result graph of PBMC-mediated killing of A-375-FAP melanoma cells promoted by antibodies Cross3×AHF21339, Cross313×AHF21339, Cross316×AHF21339, and Cross325×AHF21339 in Example 5 of the present disclosure.

FIG. 18 is a graph showing the ELISA results of binding of antibodies Cross3×AHF21339, Cross313×AHF21339, Cross316×AHF21339, and Cross325×AHF21339 to FAP protein in Example 6 of the present disclosure.

FIG. 19 is a graph showing the ELISA results of binding of Cross3×AHF21339, Cross313×AHF21339, Cross316×AHF21339, and Cross325×AHF21339 antibodies to CD3E&D protein (CD3E&CD3D) in Example 6 of the present disclosure.

FIG. 20 a graph showing the results of CD25 expression on CD4 T cells promoted by antibodies Cross 3×AHF21339 1:1, Cross313×AHF21339, Cross316×AHF21339, and Cross325×AHF21339 in Example 7 of the present disclosure.

FIG. 21 is a graph showing results of CD25 expression on CD8 T cells promoted by antibodies Cross3×AHF21339 1:1, Cross313×AHF21339, Cross316×AHF21339, and Cross325×AHF21339 in Example 7 of the present disclosure.

FIG. 22 is a graph showing results of CD69 expression on CD4 T cells promoted by antibodies Cross3×AHF21339 1:1, Cross313×AHF21339, Cross316×AHF21339, and Cross325×AHF21339 in Example 7 of the present disclosure.

FIG. 23 is a graph showing results of CD69 expression on CD8 T cells promoted by antibodies Cross3×AHF21339 1:1, Cross313×AHF21339, Cross316×AHF21339, and Cross325×AHF21339 in Example 7 of the present disclosure.

### DETAILED DESCRIPTION

Embodiments of the present disclosure will be described in detail below. The embodiments described below are illustrative, are merely used for explaining the present disclosure, and cannot be understood as a limitation to the present disclosure.

It should be noted that, terms “first” and “second” are merely used for a descriptive purpose, but cannot be understood as an indication or implication of relative importance or an implied specification of the demonstrated technical features indicated. Thus, features defined with the “first” and “second” may explicitly or implicitly include one or more features. Further, in the descriptions of the present disclosure, unless otherwise noted, the meaning of “more” refers to two or more than two.

The endpoints of the ranges and any values disclosed herein are not limited to the precise range or value, and such ranges or values should be understood to include values approximating such ranges or values. For numerical ranges, the endpoints of each range, the endpoints of each range, an endpoint value and an individual point value within each range, and individual point values can be combined with each other to obtain one or more new numerical ranges, and such numerical ranges are to be considered to be specifically disclosed herein.

For an easier understanding of the present disclosure, certain technical and scientific terms are specifically defined below. Unless obviously and clearly defined elsewhere in this document, all other technical and scientific terms used herein have the meanings commonly understood by general technical personnel in the art to which the present disclosure belongs. Abbreviations for amino acid residues are standard 3-letter and/or 1-letter codes used in the art to refer to one of the 20 commonly used L-amino acids.

In the present disclosure, the term “include/including” or “comprise/comprising” is an open-ended expression, i.e., including the contents specified in the present disclosure, but not excluding other contents.

In the present disclosure, terms “optionally” and “optional” generally mean that subsequent events or statuses may occur but not necessarily occur; and the descriptions include conditions under which the events or statuses occur, and conditions under which the events or statuses do not occur.

An antibody or antigen-binding fragment thereof according to the present disclosure is generally prepared by a biosynthetic method. According to the nucleotide sequences in the present disclosure, the coded nucleic acids according to the present disclosure may be conveniently prepared by those skilled in the art using various known methods. For example, these methods include, but not limited to: PCR, DNA artificial synthesis, etc. A specific method may refer to J. Sambrook, Molecular Cloning: A Laboratory Manual. As an implementation of the present disclosure, coded nucleic acid sequences of the present disclosure may be constructed by a method for synthesizing nucleotide sequences in segments and conducting overlap extension PCR. The antibody or the antigen fragment is numbered and defined by a Kabat numbering system. In the present disclosure, the term “antibody” refers to an immunoglobulin molecule capable of binding to a specific antigen. It includes two light chains with light molecular weight and two heavy chains with heavy molecular weight. The heavy chains (H chains) and the light chains (L chains) are connected by disulfide bonds to form a four-peptide chain molecule. An amino acid sequence at an amino terminus (N-terminus) of the peptide chain varies greatly, which is called a variable region (V region), and a carboxyl terminus (C-terminus) of the peptide chain is relatively stable with little variation, which is called a constant region (C region). The V regions of the L chain and the H chain are referred to as VL and VH, respectively. In a variable region, certain regions have a higher degree of variation in amino acid composition and arrangement, referred to as a hypervariable region (HVR), which is a location where an antigen and an antibody bind, and thus also referred to as a complementarity-determining region (CDR). There are three CDR regions in both a heavy-chain variable region and a light-chain variable region.

The antibody of the present disclosure includes a murine antibody, a chimeric antibody, and a humanized antibody, and preferably, the humanized antibody.

The term “antigen-binding fragment”, i.e. “antibody fragment” used generally refers to an antigen-binding antibody fragment and may include a portion of an intact antibody, typically an antigen-binding region or a variable region. Examples of antibody fragments include Fab, Fab′, F(ab′)2, Fv or scFv, diabody, linear antibody, single-chain antibody molecules, etc.

In the present disclosure, the term “mutant” or “variant” may refer to a molecule obtained by introducing one or more mutations of nucleotide or amino acid to any naturally occurring or engineered molecule.

In the present disclosure, the term “chimeric antibody” refers to a recombinant antibody obtained by replacing the amino acid sequence of the constant region of a monoclonal antibody from one species (e.g. mouse) with the constant region of an antibody from another species (e.g. human) using recombinant DNA technology.

In the present disclosure, the term “humanized antibody” refers to a recombinant antibody obtained by completely replacing the constant region and the non-CDR (Fv framework region (FR)) amino acid sequences of the variable region of a monoclonal antibody from one species (e.g. mouse) with the constant region and non-CDR amino acid sequences of the variable region of an antibody from another species (e.g. human) using recombinant DNA technology. That is, if the constant region of an antibody is humanized, it is referred to as a chimeric antibody, and if all of the constant region and the non-CDR amino acid sequences of the variable region are humanized, it is referred to as a humanized antibody.

In the present disclosure, the “full-length antibody” is of a tetrapeptide chain structure formed by linking two identical light chains and two identical heavy chains via interchain disulfide bonds, such as immunoglobulin G (IgG), immunoglobulin A (IgA), immunoglobulin M (IgM), immunoglobulin D (IgD) or immunoglobulin E (IgE). The immunoglobulin of the same class may also be divided into different subclasses according to amino acid compositions, such as IgG1, IgG2, IgG3, and IgG4. The light chain of the immunoglobulin is divided into K chain or 2 chain according to different constant regions.

In the present disclosure, the term “antibody affinity maturation” refers to a normal immune functional state existing in the body. In humoral immunity, a phenomenon where an average affinity of antibodies produced in a secondary response is higher than that in a primary immune response is called antibody affinity maturation. During the affinity maturation process of natural antibody, somatic hypermutation occurs predominantly in the CDR region. Through in-vitro experiments, single-site saturation mutation is performed on each site of the CDR region to obtain sufficient mutation diversity without disrupting protein structure. This approach can realize in-vitro reproduction of the somatic hypermutation of the natural antibody that are highly analogous to those occurring in vivo. A single-site saturation mutation is performed on each amino acid site in the CDR region to construct an unbiased single-site saturation mutation plasmid library of a parent antibody. Mutation hotspots with enhanced antigen-specific binding are screened using ELISA. These mutational hotspots are then combined and screened to obtain candidate antibody mutation sequences.

In the present disclosure, the term “tumor-associated fibroblasts” (also referred to as cancer-associated fibroblasts, CAFs) is one of the most predominant components in the tumor microenvironment and plays an essential role in tumor development and progression. Local tissue-resident fibroblasts and mesenchymal stem cells from bone marrow and adipose tissue are the main precursor cells of CAFs. Numerous studies have shown that CAFs do not exist as individual cells around the tumor, but interact with tumor cells, promoting tumor growth and survival and maintaining their malignant tendency.

In the present disclosure, the term “operably linked” in the present disclosure means that an exogenous gene is linked to a vector, enabling control elements in the vector, such as a transcriptional control sequence and a translational control sequence, to exert an expected effect of regulating transcription and translation of the exogenous gene. When the nucleic acid molecule is linked to the vector, the nucleic acid molecule may be directly or indirectly linked with the control elements on the vector, as long as these control elements can control translation and expression of the nucleic acid molecule. These control elements may be directly derived from the vector itself, and they may also be exogenous, that is, not from the vector. It may be understood by those skilled in the art that, the nucleic acid molecule used for coding the antibody or the antigen-binding fragment may be respectively and independently inserted into different vectors, and is commonly inserted into the same vector. Common vectors, for instance, may be plasmids, phage, etc., such as Plasmid-X.

In the present disclosure, the term “an amino acid sequence in conservative modification form” refers to an amino acid modification that does not significantly affect or alter the binding properties of the antibody including the amino acid sequence containing said amino acid, including amino acid substitutions, additions and deletions. Modifications can be introduced into the antibodies of the present disclosure by standard techniques such as site-directed mutagenesis and PCR-mediated mutagenesis. Conservative amino acid substitution refers to the substitution of amino acid residues by amino acid residues with similar side chains. Families of amino acid residues having similar side chains have been identified in the art. These families include amino acids with basic side chains, e.g. lysine, arginine, histidine, amino acids with acidic side chains, e.g. aspartic acid, glutamic acid, amino acids with uncharged polar side chains, e.g. glycine, asparagine, glutamine, serine, threonine, tyrosine, cysteine, tryptophan, amino acids with nonpolar side chains, e.g. alanine, valine, leucine, isoleucine, proline, phenylalanine, methionine, amino acids with β-branched side chains, e.g. threonine, valine, isoleucine, and amino acids with aromatic side chains, e.g. tyrosine, phenylalanine, tryptophan, histidine.

As used herein, when the term “identity” is used for describing amino acid sequences or nucleic acid sequences relative to reference sequences, and a percentage of the identical amino acids or nucleotides between two amino acid sequences or nucleic acid sequences is determined by a conventional method, for instance, see Ausubel, et al. eds. (1995), Current Protocols in Molecular Biology, Chapter 19 (Greene Publishing and Wiley-Interscience, 1995, New York); and ALIGN program (Dayhoff (1978), Atlas of Protein Sequence and Structure 5: Suppl.3 (National Biomedical Research Foundation, Washington, D.C.). Various algorithms are applicable to sequence alignment and sequence identity determination, including: a homology alignment algorithm of Needleman et al. (1970) J.Mol.Biol.48:443; a local homology algorithm of Smith et al. (1981) Adv.Appl.Math.2:482; a similarity search method of Pearson et al. (1988) Proc.Natl.Acad.Sci.85:2444; a Smith-Waterman algorithm (Meth.Mol.Biol.70:173-187 (1997)); and BLASTP, BLASTN and BLASTX algorithms (see Altschul et al. (1990) J.Mol.Biol.215:403-410). Computer programs employing these algorithms are also available, and they include, but not limited to: ALIGN or Megalign (DNASTAR) software, or WU-BLAST-2 (Altschul et al., Meth. Enzym., 266:460-480 (1996)); or GAP, BESTFIT, BLAST Altschul etc., supra, FASTA and TFASTA, available in Genetics Computing Group (GCG) package, Version 8, Madison, Wisconsin, USA; and CLUSTAL in a PC/Gene program provided by Intelligenetics, Mountain View, California.

In the present disclosure, the antibody “Cross3×AHF21339 1:1” is the same as “Cross3×AHF21339”.

According to a specific embodiment of the present disclosure, without substantially affecting antibody activity (remaining at least 95% of activity), those skilled in the art may substitute, add and/or delete one or more amino acids (such as, 1, 2, 3, 4, 5, 6, 7, 8, 9, or 10 or more) in the sequences in the present disclosure, to obtain variants of sequences of the antibody or functional fragments thereof. These variants are regarded as being included in the protection scope of the present disclosure. For example, amino acids with similar properties are substituted in a variable region. The variant sequences in the present disclosure may have at least 80%, 85%, 90%, 95%, 96%, 97%, 98% or 99% identity (or homology) to the reference sequences. The sequence identity in the present disclosure may be measured by sequence analysis software, such as a computer program BLAST with a default parameter, particularly BLASTP or TBLASTN. The amino acid sequences described in the present disclosure are all presented from the N-terminus to the C-terminus. Those skilled in the art should understand that CDR sequences analyzed by different databases may be different, but these variations should all fall within the scope of protection of the present disclosure.

According to a specific embodiment of the present disclosure, the antibody of the present disclosure may be full length (e.g. IgG1 or IgG4 antibodies) or may include only an antigen binding portion (e.g. Fab, F(ab′) 2 or scFv fragment), or may be modified to affect function. The present disclosure includes anti-CD3 antibodies having modified glycosylation patterns. In some applications, it may be useful to make modifications to remove undesired glycosylation sites, or to eliminate the presence of fucose moieties on the oligosaccharide chains, for example to enhance antibody-dependent ADCC function. In other applications, galactosylation modifications can be made to alter complement dependent cytotoxicity (CDC). After a series of modifications, the fragments described in the present disclosure still possess CD3 binding activity. Preferably, the functional fragment is composed of partial sequences in a heavy or light chain variable region of a source antibody or includes the partial sequences. The partial sequences are enough to remain binding specificity identical to the source antibody and sufficient affinity, for CD3, the affinity is preferably at least 1/100 of that of the parent antibody, and more preferably at least 1/10 thereof. The functional fragment includes at least 5 amino acids, preferably 10, 15, 25, 50, and 100 continuous amino acids in the sequence of the source antibody.

### Recombinant Antibody

The present disclosure provides a recombinant antibody. The recombinant antibody at least includes: a first binding region, in which the first binding region includes an antibody or antigen-binding fragment that specifically recognizes CD3; and a second binding region, in which the second binding region includes an antibody or antigen-binding fragment that specifically recognizes FAP. The antibody or antigen-binding fragment that specifically recognizes the CD3 in the first binding region includes an scFV region and a first Fc region. The antibody or antigen-binding fragment that specifically recognizes the FAP in the second binding region includes a Fab region and a second Fc region.

To improve the safety of a CD3 and FAP bispecific antibody, the present disclosure has obtained a CD3 and FAP recombinant antibody (bispecific antibody) having a new configuration through extensive screening and experimental verification. Further experimental results demonstrate that: the CD3 and FAP bispecific antibody of the present disclosure binds weakly to T cells, binds strongly to FAP-positive cells, and has high pro-killing activity, effectively promoting killing of the FAP-positive cells by peripheral blood mononuclear cells (PBMCs) and demonstrating satisfactory anticancer activity. In addition, the CD3 and FAP bispecific antibody has reduced capability in promoting secretion of pro-inflammatory cytokines and T cell activation, thereby possessing higher safety. Thus, the CD3 and FAP bispecific antibody has satisfactory clinical application and drug development value.

According to a specific embodiment of the present disclosure, the above recombinant antibody may further include at least one of the following additional technical features.

According to a specific embodiment of the present disclosure, in the first binding region, a C-terminus of the scFV region is linked to an N-terminus of the first Fc region.

According to a specific embodiment of the present disclosure, a C-terminus of a heavy chain variable region of the scFV region is linked to an N-terminus of a light chain variable region via a linker peptide 1, and a C-terminus of the light chain variable region is linked to the N-terminus of the first Fc region via a linker peptide 2.

According to a specific embodiment of the present disclosure, the antibody or antigen-binding fragment that specifically recognizes the FAP includes: light chain variable region CDR1, CDR2, CDR3 sequences as set forth in SEQ ID NO: 12, SEQ ID NO: 13, and SEQ ID NO: 14, respectively, and/or heavy chain variable region CDR1, CDR2, CDR3 sequences as set forth in SEQ ID NO:9, SEQ ID NO: 10, and SEQ ID NO: 11, respectively.

According to a specific embodiment of the present disclosure, the antibody or antigen-binding fragment that specifically recognizes the CD3 includes: light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 3, respectively; light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 6, respectively; or light chain variable region CDR1, CDR2, CDR3 sequences as set forth in SEQ ID NO: 4, GTN, and SEQ ID NO:8, respectively, and heavy chain variable region CDR1, CDR2, CDR3 sequences as set forth in SEQ ID NO: 1, SEQ ID NO: 2, and SEQ ID NO: 7, respectively.

According to a specific embodiment of the present disclosure, the present disclosure provides a recombinant protein including three polypeptide chains: a first polypeptide chain sequentially includes, from an N-terminus to a C-terminus, a heavy chain variable region of a CD3 antibody, a light chain variable region of the CD3 antibody, and the first Fc region; a second polypeptide chain includes a heavy chain of an FAP antibody; a third polypeptide chain includes a light chain of the FAP antibody.

According to a specific embodiment of the present disclosure, the present disclosure provides a recombinant protein including the three polypeptide chains: the first polypeptide chain sequentially includes, from the N-terminus to the C-terminus, the heavy chain variable region of the CD3 antibody (SEQ ID NO: 27, SEQ ID NO: 28, or SEQ ID NO: 30), the light chain variable region of the CD3 antibody (SEQ ID NO: 26 and SEQ ID NO: 29), and the first Fc region (SEQ ID NO: 33); the second polypeptide chain includes the heavy chain of the FAP antibody (SEQ ID NO: 19); the third polypeptide chain includes the light chain of the FAP antibody (SEQ ID NO: 20).

According to a specific embodiment of the present disclosure, an amino acid sequence of the first polypeptide chain is as set forth in SEQ ID NO: 15, SEQ ID NO: 16, or SEQ ID NO: 17, an amino acid sequence of the second polypeptide chain is as set forth in SEQ ID NO: 19, and an amino acid sequence of the third polypeptide chain is as set forth in SEQ ID NO: 20.

According to a specific embodiment of the present disclosure, the present disclosure provides a recombinant protein including four polypeptide chains: a first polypeptide chain includes a light chain of an FAP antibody; a second polypeptide chain sequentially includes, from an N-terminus to a C-terminus, a heavy chain variable region of a CD3 antibody, a CH1 region of the CD3 antibody, a heavy chain variable region of the CD3 antibody, a light chain variable region of the CD3 antibody, and the first Fc region; a second polypeptide chain includes a heavy chain of the FAP antibody; a third polypeptide chain includes a light chain of the FAP antibody.

According to a specific embodiment of the present disclosure, the present disclosure provides a recombinant protein including four polypeptide chains: the first polypeptide chain includes the light chain of the FAP antibody (SEQ ID NO: 20); the second polypeptide chain sequentially includes, from the N-terminus to the C-terminus, the heavy chain variable region of the FAP antibody (SEQ ID NO: 32), the CH1 region of the FAP antibody, the heavy chain variable region of the CD3 antibody (SEQ ID NO: 27, SEQ ID NO: 28, or SEQ ID NO: 30), the light chain variable region of the CD3 antibody (SEQ ID NO: 26 and SEQ ID NO: 29), and the first Fc region (SEQ ID NO: 33); the second polypeptide chain includes the heavy chain of the FAP antibody (SEQ ID NO: 19); the third polypeptide chain includes the light chain of the FAP antibody (SEQ ID NO: 20).

According to an embodiment of the present disclosure, the present disclosure has screened out two CD3-FAP bispecific antibodies having different configurations. Even when the antigen-binding domains are the same, antigen-binding capacities of antibodies having different configurations are not exactly the same. One objective of the present disclosure is to screen out a superior CD3-FAP bispecific antibody with a better effect in promoting killing of tumors by immune cells, which includes the recombinant protein including the three polypeptide chains: the first polypeptide chain sequentially includes, from the N-terminus to the C-terminus, the heavy chain variable region of the CD3 antibody, the light chain variable region of the CD3 antibody, and the first Fc region; the second polypeptide chain includes the heavy chain of the FAP antibody; the third polypeptide chain includes the light chain of the FAP antibody. This antibody not only has a better ability to promote killing of non-small cell lung cancer HCC827 cells by immune cells, but also has a satisfactory effect when killing other B7H7-positive cells.

According to an embodiment of the present disclosure, the FAP is specifically expressed on the surface of tumor fibroblasts, which is a part of the tumor microenvironment. A purpose of anticancer is achieved by destroying the tumor microenvironment. It should be noted that the anti-FAP antibody provided by the present disclosure can act on any tumor in which the FAP is expressed on tumor fibroblasts in the tumor microenvironment. The tumor is not limited here, and may be any one or more of lung cancer, liver cancer, ovarian cancer, cervical cancer, skin cancer, bladder cancer, colon cancer, breast cancer, melanoma, glioma, kidney cancer, gastric cancer, esophageal cancer, oral squamous cell carcinoma, and head-and-neck cancer.

### Nucleic Acid Molecule, Recombinant Vector, and Immunoconjugate

In a process of preparing or obtaining these antibodies, nucleic acid molecules expressing these antibodies can be used to link with different vectors, and then expressed in different cells to obtain corresponding antibodies.

Therefore, the present disclosure also provides an isolated nucleic acid encoding the above recombinant antibody, antibody, or antigen-binding fragment, and a recombinant vector and transformant including the nucleic acid. The nucleic acid molecule encodes the above recombinant antibody, antibody, or antigen-binding fragment. The nucleic acid is preferably an expression cassette obtained by means of genetic engineering.

The recombinant vector can refer to a cloning vector or an expression vector, and can be obtained by operably linking the nucleic acid with a commercially available vector (such as a plasmid or viral vector). Commonly used plasmids include pSeTag2, PEE14, pMH3, etc.

In some preferred embodiments, the nucleic acid molecule is species-optimized for easier expression in mammalian cells.

The present disclosure also provides an expression vector including the above isolated nucleic acid molecule. When the above isolated polynucleotide is linked to a vector, the polynucleotide may be directly or indirectly connected with control elements on the vector, as long as these control elements can control translation and expression of the polynucleotide. These control elements may be directly from the vector, or they may also be exogenous, that is, not from the vector. The polynucleotide just needs to be operably linked to the control elements.

The immunoconjugate provided by the present disclosure includes a therapeutic agent and the recombinant antibody, antibody, or antigen binding fragment described above conjugated to the therapeutic agent. The recombinant antibody, antibody, or antigen binding fragment can be conjugated to the therapeutic agent in a conventional manner.

The composition provided by the present disclosure includes the recombinant antibody, antibody, or antigen binding fragment described above, and/or the immunoconjugate described above, and a pharmaceutically acceptable carrier. In certain embodiments, the composition includes combinations separated temporally and/or spatially, as long as they are capable of acting together to achieve the objectives of the present disclosure. For example, components contained in the composition may be administered to a subject as a whole or separately. When the components contained in the composition are separately administered to the subject, the respective components may be simultaneously or sequentially administered to the subject.

### Recombinant Cell

The present disclosure provides a recombinant cell. The recombinant cell carries the aforementioned nucleic acid or the aforementioned vector or transformant, or expresses the aforementioned recombinant antibody. According to an embodiment of the present disclosure, the cell is obtained by transfection or transformation of the vector or transformant, and can thus efficiently express the aforementioned recombinant antibody under suitable conditions.

According to a specific embodiment of the present disclosure, the above recombinant cell may further include at least one of the following additional technical features.

According to an embodiment of the present disclosure, the cell is a prokaryotic cell, a eukaryotic cell, or a bacteriophage.

According to an embodiment of the present disclosure, the prokaryotic cell is Escherichia coli, Bacillus subtilis, Streptomyces, or Proteus mirabilis.

According to an embodiment of the present disclosure, the eukaryotic cell is fungus, an insect cell, a plant cell, or a mammalian cell.

According to an embodiment of the present disclosure, the fungus is Pichia pastoris, Saccharomyces cerevisiae, Schizosaccharomyces pombe, or Trichoderma.

According to an embodiment of the present disclosure, the insect cell is a Spodoptera frugiperda cell. According to an embodiment of the present disclosure, the plant cell is Nicotiana tabacum cell.

According to an embodiment of the present disclosure, the mammalian cell is a BHK cell, a CHO cell, a COS cell, a myeloma cell, or a human embryonic kidney 293 cell, and does not include an animal germ cell, fertilized egg, or embryonic stem cell.

According to an embodiment of the present disclosure, the cells are mammalian cells.

According to an embodiment of the present disclosure, the cells are BHK cells, CHO cells, COS cells, or NSO cells.

It should be noted that “suitable conditions” as used in the description of the present application refers to conditions suitable for the expression of the recombinant antibody, antibody, or antigen binding fragment described herein. Those skilled in the art will readily appreciate that conditions suitable for the expression of the recombinant antibody, antibody, or antigen binding fragment include, but are not limited to, suitable transformation or transfection manners, suitable transformation or transfection conditions, healthy host cell state, suitable host cell density, suitable cell culture environment, and suitable cell culture time. The “suitable conditions” are not particularly limited, and one skilled in the art can optimize the optimal conditions for expression of the recombinant antibody, antibody, or antigen binding fragment according to the specific environments of the laboratory.

### Composition

The present disclosure provides a composition. The composition includes the aforementioned recombinant antibody, the aforementioned nucleic acid, the aforementioned vector or transformant, or the aforementioned cell. According to an embodiment of the present disclosure, the recombinant antibody can effectively promote killing of tumor cells by the PBMCs and have the anticancer activity. In addition, the recombinant antibody can produce fewer pro-inflammatory cytokines, has higher safety, and possesses satisfactory clinical application value and drug development value. Thus, the obtained composition can be further used for preventing and/or treating a CD3-mediated related disease and/or an FAP-mediated related disease.

### Medicament

The present disclosure provides a medicament. The medicament includes the aforementioned recombinant antibody, the aforementioned nucleic acid, the aforementioned vector or transformant, the aforementioned cell, or the aforementioned composition. According to an embodiment of the present disclosure, the recombinant antibody can effectively promote the killing of the tumor cells by the PBMCs and have the anticancer activity. In addition, the recombinant antibody can produce fewer pro-inflammatory cytokines, has higher safety, and possesses the satisfactory clinical application value and drug development value. Thus, the obtained medicament can be further used for preventing and/or treating the CD3-mediated related disease and/or the FAP-mediated related disease.

According to an embodiment of the present disclosure, the above medicament may further include at least one of the following additional technical features.

According to an embodiment of the present disclosure, the above medicament further includes a pharmaceutically acceptable adjuvant.

According to an embodiment of the present disclosure, the adjuvant includes one or more pharmaceutically acceptable excipients, a diluent, a stabilizer, or a carrier.

According to an embodiment of the present disclosure, the medicament is an injection.

It should be noted that, the medicament includes combinations separated temporally and/or spatially, as long as they are capable of acting together to achieve objectives of the present disclosure. For example, components contained in the medicament may be administered to a subject as a whole or separately. When the components contained in the medicament are separately administered to the subject, the respective components may be simultaneously or sequentially administered to the subject.

The medicament of the present disclosure contains a safe and effective amount of the active ingredient of the present disclosure and a pharmaceutically acceptable adjuvant. Such excipients include but are not limited to saline, buffer, glucose, water, glycerol, ethanol, and combinations thereof. Generally, the pharmaceutical preparation should be matched with the administration mode, and the dosage form of the medicament of the present disclosure can be an injection, an oral preparation (tablet, capsule, and oral liquid), a transdermal agent, or a sustained-release agent. They are prepared by conventional methods, for example, using physiological saline or aqueous solutions containing glucose and other adjuvants. The medicament is preferably manufactured under sterile conditions.

The effective amount of the described active ingredient in the present disclosure can vary depending on the mode of administration and the severity of the disease to be treated. The selection of the optimal effective amount can be determined by ordinary technicians in the field based on various factors (such as through clinical trials). Such factors include, but are not limited to pharmacokinetic parameters of the active ingredient such as bioavailability, metabolism, half-life, and the like; the severity of the disease of the patient to be treated, the weight of the patient, the immune status of the patient, the route of administration, etc. For example, several divided doses may be administered daily or the dose may be proportionally reduced as indicated by the exigencies of the therapeutic situation.

Pharmaceutically acceptable adjuvants described herein include but are not limited to water, saline, liposomes, lipids, proteins, protein-antibody conjugates, peptides, cellulose, nanogel, or combinations thereof. The selection of carriers should be matched with the administration route, which is well-known to ordinary technical personnel in this field.

### Kit

The present disclosure provides a kit. The kit includes the aforementioned recombinant antibody, the aforementioned nucleic acid molecule, the aforementioned vector or transformant, or the aforementioned cell. According to an embodiment of the present disclosure, the kit can bind to CD3 protein and/or FAP protein, and can effectively identify the CD3 protein and/or the FAP protein.

As mentioned above, the recombinant antibody according to an embodiment of the present disclosure can specifically bind to the CD3 and the FAP. CD3 protein and/or FAP protein-related kits developed by using this property can be used for research related to the CD3 protein and/or the FAP protein, such as for detection and/or enrichment and/or separation and purification of CD3 and/or FAP proteins of humans or other mammals.

The kit can effectively detect, enrich, or separate and purify CD3 and/or FAP proteins in biological samples for further scientific research, such as qualitative or quantitative detection of CD3 and/or FAP protein molecules in biological samples. More specifically, it can be used in kits for detection involving the specific binding properties of CD3 protein and/or FAP protein to antibodies, such as immunoblotting, and immunoprecipitation. These kits may include any one or more of the following: antagonist, the recombinant antibody of the present disclosure, or drug reference material; a protein purification column; an immunoglobulin affinity purification buffer; an assay diluent for cells. The recombinant antibody of the present disclosure can be used for different types of diagnostic tests, e.g. in vitro or in vivo detection of a wide variety of diseases, or the presence of medicaments, toxins or other proteins, etc. For example, the CD3-mediated related disease and/or the FAP-mediated related disease can be tested by testing serum or blood from the subject.

### Use in the Manufacture of a Kit

The present disclosure provides use of the aforementioned recombinant antibody, the aforementioned nucleic acid molecule, the aforementioned vector or transformant, or the aforementioned cell in the manufacture of a kit for detecting CD3 and/or FAP.

As mentioned above, the recombinant antibody according to an embodiment of the present disclosure can specifically bind to the CD3 and the FAP. Therefore, the recombinant antibody can be used for detecting the CD3 and/or the FAP. Further, the recombinant antibody can be used for preparing CD3 and/or FAP-related kits and for scientific research, such as qualitative or quantitative detection of CD3 and/or FAP protein molecules in biological samples. More specifically, it can be used in kits for detection involving the specific binding properties of CD3 and/or FAP to antibodies, such as immunoblotting, and immunoprecipitation. These kits may include any one or more of the following: antagonist, the recombinant antibody of the present disclosure, or drug reference material; a protein purification column; an immunoglobulin affinity purification buffer; an assay diluent for cells. The recombinant antibody of the present disclosure can be used for different types of diagnostic tests, e.g. in vitro or in vivo detection of a wide variety of diseases or the presence of medicaments, toxins or other proteins, etc. For example, the CD3-mediated related disease and/or the FAP-mediated related disease can be tested by testing serum or blood from the subject.

### Use in the Manufacture of a Medicament

The present disclosure provides use of the aforementioned recombinant antibody, the aforementioned nucleic acid molecule, the aforementioned vector or transformant, the aforementioned cell, or the aforementioned composition in the manufacture of a medicament for preventing and/or treating a CD3-mediated related disease and/or an FAP-mediated related disease. According to an embodiment of the present disclosure, the recombinant antibody of the present disclosure and the corresponding nucleic acid, vector or transformant, or composition can be further prepared into the medicament. The medicament can be used for preventing or treating the CD3-mediated related disease and/or the FAP-mediated related disease in clinical practice.

According to an embodiment of the present disclosure, the above use may also include at least one of the following additional technical features.

According to an embodiment of the present disclosure, the CD3-mediated related disease includes an autoimmune disease.

According to an embodiment of the present disclosure, the autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

According to an embodiment of the present disclosure, the FAP-mediated related disease is cancer, a disease caused by transplant rejection, an autoimmune disease, or an infectious disease.

According to an embodiment of the present disclosure, the cancer is at least one of lung cancer, liver cancer, ovarian cancer, cervical cancer, skin cancer, bladder cancer, colon cancer, breast cancer, glioma, kidney cancer, gastric cancer, esophageal cancer, oral squamous cell carcinoma, or head-and-neck cancer.

Use and Method for Preventing and/or Treating a Disease

The present disclosure provides use of the aforementioned recombinant antibody, the aforementioned nucleic acid molecule, the aforementioned vector or transformant, the aforementioned cell, the aforementioned composition, or the aforementioned medicament in preventing and/or treating a CD3-mediated related disease and/or an FAP-mediated related disease. As mentioned above, the recombinant antibody can effectively promote the killing of the FAP-positive cells by the PBMCs and demonstrating the satisfactory anticancer activity. In addition, the recombinant antibody has reduced capability in promoting secretion of the pro-inflammatory cytokines and the T cell activation, thereby possessing higher safety. Thus, the recombinant antibody, and substances capable of expressing and obtaining the antibody or antigen-binding fragment thereof under suitable conditions, or substances including the recombinant antibody, can all be used for preventing and/or treating the CD3-mediated related disease and/or the FAP-mediated related disease.

According to an embodiment of the present disclosure, the above use for treating the disease may further include at least one of the following additional technical features.

In some specific embodiments, the CD3-mediated related disease includes an autoimmune disease.

In some specific embodiments, the autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

In some specific embodiments, the FAP-mediated disease includes a tumor. The tumor-associated fibroblasts of the tumor is FAP-positive.

In some specific embodiments, a disease caused by the tumor includes lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

The present disclosure provides the aforementioned recombinant antibody, the aforementioned nucleic acid molecule, the aforementioned vector or transformant, the aforementioned cell, or the aforementioned pharmaceutical composition, for use in preventing and/or treating the CD3-mediated related disease and/or the FAP-mediated related disease. As mentioned above, the recombinant antibody can effectively promote the killing of the FAP-positive cells by the PBMCs and demonstrating the satisfactory anticancer activity. In addition, the recombinant antibody has reduced capability in promoting the secretion of the pro-inflammatory cytokines and the T cell activation, thereby possessing higher safety. Thus, the recombinant antibody, and the substances capable of expressing and obtaining the antibody or antigen-binding fragment thereof under suitable conditions, or the substances including the recombinant antibody, can all be used for preventing and/or treating the CD3-mediated related disease and/or the FAP-mediated related disease.

According to an embodiment of the present disclosure, the above use for treating and/or preventing the disease may further include at least one of the following additional technical features.

In some specific embodiments, the CD3-mediated related disease includes an autoimmune disease.

In some specific embodiments, the autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

In some specific embodiments, the FAP-mediated disease includes a tumor. The tumor-associated fibroblasts of the tumor is FAP-positive.

In some specific embodiments, a disease caused by the tumor includes lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

The present disclosure provides a method for preventing and/or treating a CD3-mediated related disease and/or an FAP-mediated related disease. According to an embodiment of the present disclosure, the method includes: administering to a subject a pharmaceutically acceptable amount of the aforementioned recombinant antibody, the aforementioned nucleic acid molecule, the aforementioned vector or transformant, the aforementioned cell, the aforementioned composition, or the aforementioned medicament. As mentioned above, the recombinant antibody can effectively promote the killing of the FAP-positive cells by the PBMCs and demonstrating the satisfactory anticancer activity. In addition, the recombinant antibody has reduced capability in promoting the secretion of the pro-inflammatory cytokines and the T cell activation, thereby possessing higher safety. Thus, the recombinant antibody, and the substances capable of expressing and obtaining the antibody or antigen-binding fragment thereof under suitable conditions, or the substances including the recombinant antibody, can all effectively prevent and/or treat the CD3-mediated related disease and/or the FAP-mediated related disease. The method according to the embodiments of the present disclosure can effectively prevent and/or treat the CD3-mediated related disease and/or the FAP-mediated related disease.

It should be noted that the terms “subject,” “individual,” and “patient” may be used interchangeably herein and refer to a mammal being evaluated for treatment and/or being treated. In an embodiment, the mammal is a human. The terms “subject,” “individual,” and “patient” include, but are not limited to, individuals with cancer, individuals with autoimmune diseases, individuals with pathogen infection, and the like. The subject may be a human, but also includes other mammals, especially mammals that can be used as laboratory models for human diseases, such as mice and rats.

The effective amount of the recombinant antibody, antibody or antigen-binding fragment thereof, conjugate, nucleic acid, vector or transformant, composition, or medicament described in the present disclosure can vary depending on the mode of administration and the severity of the disease to be treated. The selection of the optimal effective amount can be determined by ordinary technicians in the field based on various factors (such as through clinical trials). Such factors include, but are not limited to pharmacokinetic parameters of the active ingredient such as bioavailability, metabolism, half-life, and the like; the severity of the disease of the patient to be treated, the weight of the patient, the immune status of the patient, the route of administration, etc. For example, several divided doses may be administered daily or the dose may be proportionally reduced as indicated by the exigencies of the therapeutic situation.

According to an embodiment of the present disclosure, the above method for treating and/or preventing the disease may further include at least one of the following additional technical features.

In some specific embodiments, the CD3-mediated related disease includes an autoimmune disease.

In some specific embodiments, the autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

In some specific embodiments, the FAP-mediated related disease includes a tumor. The tumor-associated fibroblasts of the tumor is FAP-positive.

In some specific embodiments, a disease caused by the tumor includes lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

### Detection and Diagnostic Use

The present disclosure provides use of the aforementioned recombinant antibody, the aforementioned polynucleotide, or the aforementioned kit in detecting CD3 and/or FAP in a to-be-tested sample. As mentioned above, the recombinant antibody according to the embodiments of the present disclosure is capable of specifically binding to the CD3 and/or the FAP. Therefore, the recombinant antibody and related products thereof can be used to detect the CD3 and/or the FAP, for example, for scientific research, such as qualitative or quantitative detection of CD3 and/or FAP protein molecules in the to-be-tested sample.

In some specific embodiments, the to-be-tested sample includes at least one of: a tissue, a cell, blood, serum, plasma, saliva, sweat, feces, or urine.

The present disclosure provides the use of the aforementioned recombinant antibody, the aforementioned polynucleotide, or the aforementioned kit in diagnosing whether a subject is suffering from a CD3-mediated related disease and/or an FAP-mediated related disease. As mentioned above, the recombinant antibody according to the embodiments of the present disclosure can effectively bind to CD3 and/or FAP. Therefore, the recombinant antibody can be used to detect a level of CD3 and/or FAP, and CD3 and/or FAP-mediated various diseases. In this way, whether the subject suffers from the CD3-mediated related disease and/or the FAP-mediated related disease can be determined based on the level of the CD3 and/or the FAP contained in a biological sample from the subject. In addition, a content of the CD3 and/or the FAP in the to-be-tested sample from the subject can also be monitored by using the above substances. That is, the above substances can also be used for disease staging of the subject suffering from the CD3-mediated related disease and/or the FAP-mediated related disease, and can also be used for prognosis evaluation of the CD3-mediated related disease and/or the FAP-mediated related disease.

According to an embodiment of the present disclosure, the above use in diagnosing the disease may further include at least one of the following additional technical features.

In some specific embodiments, the CD3-mediated related disease includes an autoimmune disease.

In some specific embodiments, the autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

In some specific embodiments, the FAP-mediated disease includes a tumor. The tumor-associated fibroblasts of the tumor is FAP-positive.

In some specific embodiments, a disease caused by the tumor includes lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

### Detection and Diagnostic Method

The present disclosure provides a method for detecting CD3 and/or FAP. According to an embodiment of the present disclosure, the method includes: detecting a to-be-tested sample using the aforementioned recombinant antibody, the aforementioned polynucleotide, or the aforementioned kit.

In some specific embodiments, the to-be-tested sample includes at least one of: a tissue, a cell, blood, serum, plasma, saliva, sweat, feces, or urine.

The present disclosure provides a method for diagnosing whether a subject is suffering from a CD3-mediated related disease and/or an FAP-mediated related disease. According to an embodiment of the present disclosure, the method includes: detecting the to-be-tested sample from the subject using the aforementioned recombinant antibody, the aforementioned polynucleotide, or the aforementioned kit. As mentioned above, the recombinant antibody according to the embodiments of the present disclosure can effectively bind to CD3 and/or FAP. Therefore, the recombinant antibody can be used to detect the level of CD3 and/or FAP, and CD3 and/or FAP-mediated various diseases. In this way, whether the subject suffers from the CD3-mediated related disease and/or the FAP-mediated related disease can be determined based on the level of the CD3 and/or the FAP contained in the biological sample from the subject. In addition, the content of the CD3 and/or the FAP in the to-be-tested sample from the subject can also be monitored by using the above substances. That is, the above substances can also be used for disease staging of the subject suffering from the CD3-mediated related disease and/or the FAP-mediated related disease, and can also be used for prognosis evaluation of the CD3-mediated related disease and/or the FAP-mediated related disease.

According to an embodiment of the present disclosure, the above method for diagnosing the disease may further include at least one of the following additional technical features.

In some specific embodiments, the CD3-mediated related disease includes an autoimmune disease.

In some specific embodiments, said autoimmune disease includes at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis.

In some specific embodiments, the FAP-mediated disease includes a tumor. The tumor-associated fibroblasts of the tumor is FAP-positive.

In some specific embodiments, a disease caused by the tumor includes lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

In some specific embodiments, the to-be-tested sample includes at least one of: a tissue, a cell, blood, serum, plasma, saliva, sweat, feces, or urine.

Nucleic acids encoding the heavy chain and/or light chain of the antibody of the present disclosure fall within the scope of the present disclosure. Based on the amino acid sequences of the heavy chain and/or light chain, those skilled in the art can readily obtain the corresponding nucleic acid sequences, as shown in Table 1.

TABLE 1

| SEQ | | |
|---|---|---|
| ID | | |
| NO: | Sequence | Description |
| 1 | GFTFNTYA | Cross313/316/325 |
| | | HCDR1 |
| 2 | IRSKYNNYAT | Cross313/316/325 |
| | | HCDR2 |
| 3 | VRHGNFGNSYQSWFAY | Cross313 HCDR3 |
| 4 | TGAVTTSNY | Cross313/316/325 |
| | | LCDR1 |
| GTN | GTN | Cross313/316/325 |
| | | LCDR2 |
| 5 | ALWYSNLWV | Cross313/316 LCDR3 |
| 6 | VRHGNFGNSYVSWFLY | Cross316 HCDR3 |
| 7 | VRHGNFGNSYVSWFAY | Cross325 HCDR3 |
| 8 | ALWYSNDWV | Cross325 LCDR3 |
| 9 | ENIIH | AHF21339 HCDR1 |
| 10 | WFHPGSGSIKYNEKFKD | AHF21339 HCDR2 |
| 11 | HKGTGRGAMDY | AHF21339 HCDR3 |
| 12 | RASKSLSYGAYSYMH | AHF21339 LCDR1 |
| 13 | LASNLES | AHF21339 LCDR2 |
| 14 | QHSRELPYT | AHF21339 LCDR3 |
| 15 | EVQLLESGGGLVQPGGSLKLSCAASGFTFNTYAMNWVRQAP | First binding region of |
| | GKGLEWVARIRSKYNNYATYYADSVKDRFTISRDDSKNTAY | configuration A |
| | LQMNNLKTEDTAVYYCVRHGNFGNSYQSWFAYWGQGTLVT | (Cross313 × AHF21339 |
| | VSSGGGGSGGGGSGGGGSELVVTQEPSLTVSPGGTVTLTCRSS | |
| | TGAVTTSNYANWVQQKPGQAPRGLIGGTNKRAPGTPARFSGS | |
| | LLGGKAALTLSGVQPEDEAEYYCALWYSNLWVFGGGTKLTV | |
| | LGGGGSPKSSDKTHTCPPCPAPEAAGGPSVFLFPPKPKDTLMIS | |
| | RTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCREEMTKNQVSLWCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| 16 | EVQLLESGGGLVQPGGSLKLSCAASGFTFNTYAMNWVRQAP | First binding region of |
| | GKGLEWVARIRSKYNNYATYYADSVKDRFTISRDDSKNTAY | configuration A |
| | LQMNNLKTEDTAVYYCVRHGNFGNSYVSWFLYWGQGTLVT | (Cross316 × AHF21339) |
| | VSSGGGGSGGGGSGGGGSELVVTQEPSLTVSPGGTVTLTCRSS | |
| | TGAVTTSNYANWVQQKPGQAPRGLIGGTNKRAPGTPARFSGS | |
| | LLGGKAALTLSGVQPEDEAEYYCALWYSNLWVFGGGTKLTV | |
| | LGGGGSPKSSDKTHTCPPCPAPEAAGGPSVFLFPPKPKDTLMIS | |
| | RTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCREEMTKNQVSLWCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| 17 | EVQLLESGGGLVQPGGSLKLSCAASGFTFNTYAMNWVRQAP | First binding region of |
| | GKGLEWVARIRSKYNNYATYYADSVKDRFTISRDDSKNTAY | configuration A |
| | LQMNNLKTEDTAVYYCVRHGNFGNSYVSWFAYWGQGTLVT | (Cross325 × AHF21339 |
| | VSSGGGGSGGGGSGGGGSELVVTQEPSLTVSPGGTVTLTCRSS | ) |
| | TGAVTTSNYANWVQQKPGQAPRGLIGGTNKRAPGTPARFSGS | |
| | LLGGKAALTLSGVQPEDEAEYYCALWYSNDWVFGGGTKLTV | |
| | LGGGGSPKSSDKTHTCPPCPAPEAAGGPSVFLFPPKPKDTLMIS | |
| | RTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCREEMTKNQVSLWCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| 18 | EVQLLESGGGLVQPGGSLKLSCAASGFTFNTYAMNWVRQAP | First binding region of |
| | GKGLEWVARIRSKYNNYATYYADSVKDRFTISRDDSKNTAY | configuration A |
| | LQMNNLKTEDTAVYYCVRHGNFGNSYVSWFAYWGQGTLVT | (Cross3 × AHF21339 1:1) |
| | VSSGGGGSGGGGSGGGGSELVVTQEPSLTVSPGGTVTLTCRSS | |
| | TGAVTTSNYANWVQQKPGQAPRGLIGGTNKRAPGTPARFSGS | |
| | LLGGKAALTLSGVQPEDEAEYYCALWYSNLWVFGGGTKLTV | |
| | LGGGGSPKSSDKTHTCPPCPAPEAAGGPSVFLFPPKPKDTLMIS | |
| | RTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCREEMTKNQVSLWCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| 19 | QVQLVQSGAEVKKPGASVKVSCKASGYTFTENIIHWVRQAPG | AHF21339 heavy |
| | QGLEWIGWFHPGSGSIKYNEKFKDRVTMTADTSISTAYMELS | chain |
| | RLRSDDTAVYYCARHKGTGRGAMDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVCTLPPSREEMTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 20 | DIVLTQSPASLAVSPGQRATITCRASKSLSYGAYSYMHWYQQ | AHF21339 light |
| | KPGQPPKLLIYLASNLESGVPARFSGSGSGTDFTLTINPVEAND | chain |
| | AANYYCQHSRELPYTFGGGTKLEIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| 21 | QVQLVQSGAEVKKPGASVKVSCKASGYTFTENIIHWVRQAPG | Configuration B: |
| | QGLEWIGWFHPGSGSIKYNEKFKDRVTMTADTSISTAYMELS | heavy chain variable |
| | RLRSDDTAVYYCARHKGTGRGAMDYWGQGTLVTVSSASTK | region and CH1 region |
| | GPSVFPLAPSSKSTSGGTAALGCLVKDYFPEPVTVSWNSGALT | of FAP antibody +scFv |
| | SGVHTFPAVLQSSGLYSLSSVVTVPSSSLGTQTYICNVNHKPS | region of CD3 |
| | NTKVDKKVEPKSCDKTHTSPPSPEVQLLESGGGLVQPGGSLK | antibody + first Fc |
| | LSCAASGFTFNTYAMNWVRQAPGKGLEWVARIRSKYNNYAT | region in |
| | YYADSVKDRFTISRDDSKNTAYLQMNNLKTEDTAVYYCVRH | Cross3 × AHF21339 2:1 |
| | GNFGNSYVSWFAYWGQGTLVTVSSGGGGSGGGGSGGGGSEL | antibody |
| | VVTQEPSLTVSPGGTVTLTCRSSTGAVTTSNYANWVQQKPGQ | |
| | APRGLIGGTNKRAPGTPARFSGSLLGGKAALTLSGVQPEDEAE | |
| | YYCALWYSNLWVFGGGTKLTVLGGGGSPKSSDKTHTCPPCP | |
| | APEAAGGPSVFLFPPKPKDTLMISRTPEVTCVVVDVSHEDPEV | |
| | KFNWYVDGVEVHNAKTKPREEQYNSTYRVVSVLTVLHQDW | |
| | LNGKEYKCKVSNKALPAPIEKTISKAKGQPREPQVYTLPPCRE | |
| | EMTKNQVSLWCLVKGFYPSDIAEWESNGQPENNYKTTPPV | |
| | LDSDGSFFLYSKLTVDKSRWQQGNVFSCSVMHEALHNHYTQ | |
| | KSLSLSPGK | |
| 22 | EVQLVESGGGLVQPGGSLRLSCAASGYSFTGYTMNWVRQAP | First binding region of |
| | GKGLEWVALINPYKGVSTYNQKFKDRFTISVDKSKNTAYLQ | configuration A |
| | MNSLRAEDTAVYYCARSGYYGDSDWYFDVWGQGTLVTVSS | (UCHT1 × AHF21339 1:1) |
| | GGGGSGGGGSGGGGSDIQMTQSPSSLSASVGDRVTITCRASQ | |
| | DIRNYLNWYQQKPGKAPKLLIYYTSRLESGVPSRFSGSGSGTD | |
| | YTLTISSLQPEDFATYYCQQGNTLPWTFGQGTKVEIKGGGGSP | |
| | KSSDKTHTCPPCPAPEAAGGPSVFLFPPKPKDTLMISRTPEVTC | |
| | VVVDVSHEDPEVKFNWYVDGVEVHNAKTKPREEQYNSTYR | |
| | VVSVLTVLHQDWLNGKEYKCKVSNKALPAPIEKTISKAKGQP | |
| | REPQVYTLPPCREEMTKNQVSLWCLVKGFYPSDIAVEWESNG | |
| | QPENNYKTTPPVLDSDGSFFLYSKLTVDKSRWQQGNVFSCSV | |
| | MHEALHNHYTQKSLSLSPGK | |
| 23 | QVQLVQSGAEVKKPGASVKVSCKASGYTFTENIIHWVRQAPG | First binding region of |
| | QGLEWIGWFHPGSGSIKYNEKFKDRVTMTADTSISTAYMELS | configuration B: heavy |
| | RLRSDDTAVYYCARHKGTGRGAMDYWGQGTLVTVSSASTK | chain variable region |
| | GPSVFPLAPSSKSTSGGTAALGCLVKDYFPEPVTVSWNSGALT | and CHI region of |
| | SGVHTFPAVLQSSGLYSLSSVVTVPSSSLGTQTYICNVNHKPS | FAP antibody + scFv |
| | NTKVDKKVEPKSCDKTHTSPPSPEVQLVESGGGLVQPGGSLR | region of CD3 |
| | LSCAASGYSFTGYTMNWVRQAPGKGLEWVALINPYKGVSTY | antibody + first Fc |
| | NQKFKDRFTISVDKSKNTAYLQMNSLRAEDTAVYYCARSGY | region in |
| | YGDSDWYFDVWGQGTLVTVSSGGGGSGGGGSGGGGSDIQM | UCHT1 × AHF21339 2:1 |
| | TQSPSSLSASVGDRVTITCRASQDIRNYLNWYQQKPGKAPKLL | antibody |
| | IYYTSRLESGVPSRFSGSGSGTDYTLTISSLQPEDFATYYCQQG | |
| | NTLPWTFGQGTKVEIKGGGGSPKSSDKTHTCPPCPAPEAAGG | |
| | PSVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPCREEMTKNQ | |
| | VSLWCLVKGFYPSDIAVEWESNGQPENNYKTTPPVLDSDGSF | |
| | FLYSKLTVDKSRWQQGNVFSCSVMHEALHNHYTQKSLSLSP | |
| | GK | |
| 24 | MKTWVKIVFGVATSAVLALLVMCIVLRPSRVHNSEENTMRA | Human FAP |
| | LTLKDILNGTFSYKTFFPNWISGQEYLHQSADNNIVLYNIETG | |
| | QSYTILSNRTMKSVNASNYGLSPDRQFVYLESDYSKLWRYSY | |
| | TATYYIYDLSNGEFVRGNELPRPIQYLCWSPVGSKLAYVYQN | |
| | NIYLKQRPGDPPFQITFNGRENKIFNGIPDWVYEEEMLATKYA | |
| | LWWSPNGKFLAYAEFNDTDIPVIAYSYYGDEQYPRTINIPYPK | |
| | AGAKNPVVRIFIIDTTYPAYVGPQEVPVPAMIASSDYYFSWLT | |
| | WVTDERVCLQWLKRVQNVSVLSICDFREDWQTWDCPKTQE | |
| | HIEESRTGWAGGFFVSTPVFSYDAISYYKIFSDKDGYKHIHYIK | |
| | DTVENAIQITSGKWEAINIFRVTQDSLFYSSNEFEEYPGRRNIY | |
| | RISIGSYPPSKKCVTCHLRKERCQYYTASFSDYAKYYALVCYG | |
| | PGIPISTLHDGRTDQEIKILEENKELENALKNIQLPKEEIKKLEV | |
| | DEITLWYKMILPPQFDRSKKYPLLIQVYGGPCSQSVRSVFAVN | |
| | WISYLASKEGMVIALVDGRGTAFQGDKLLYAVYRKLGVYEV | |
| | EDQITAVRKFIEMGFIDEKRIAIWGWSYGGYVSSLALASGTGL | |
| | FKCGIAVAPVSSWEYYASVYTERFMGLPTKDDNLEHYKNSTV | |
| | MARAEYFRNVDYLLIHGTADDNVHFQNSAQIAKALVNAQVD | |
| | FQAMWYSDQNHGLSGLSTNHLYTHMTHFLKQCFSLSD | |
| 25 | ATGAAGACATGGGTCAAGATCGTATTCGGAGTAGCTACCT | Human FAP base |
| | CAGCAGTGCTTGCCCTGCTCGTGATGTGCATCGTTCTGAGG | sequence |
| | CCATCTCGAGTTCATAATAGCGAAGAAAACACAATGCGTG | |
| | CTCTCACTCTGAAGGATATTCTCAACGGTACCTTTTCTTATA | |
| | AGACATTCTTCCCTAACTGGATCAGTGGTCAGGAGTATTTG | |
| | CACCAGTCAGCTGATAACAACATAGTCTTGTACAACATTGA | |
| | GACTGGGCAGTCATACACAATCCTGAGTAATAGGACTATG | |
| | AAAAGCGTCAATGCTAGTAATTACGGACTGTCCCCAGATC | |
| | GCCAGTTCGTGTATCTGGAAAGTGACTACTCTAAGCTCTGG | |
| | AGATACTCTTACACCGCTACCTATTACATATACGATCTGTC | |
| | TAACGGCGAGTTTGTGAGAGGCAATGAACTTCCCAGACCA | |
| | ATTCAATACCTGTGCTGGAGCCCAGTAGGAAGCAAACTGG | |
| | CTTATGTGTATCAGAACAATATCTATCTGAAGCAGAGGCCT | |
| | GGAGATCCTCCTTTCCAGATAACTTTCAATGGGCGGGAAAA | |
| | CAAGATCTTTAATGGCATTCCCGACTGGGTCTATGAGGAAG | |
| | AGATGCTGGCCACCAAGTACGCTCTGTGGTGGAGCCCCAA | |
| | CGGAAAGTTTCTCGCCTACGCTGAATTCAACGACACCGATA | |
| | TACCAGTGATCGCCTATAGTTACTATGGTGATGAGCAGTAT | |
| | CCAAGAACTATTAATATACCTTATCCCAAGGCTGGAGCCAA | |
| | AAACCCAGTGGTACGAATTTTTATCATCGATACCACCTACC | |
| | CAGCTTACGTGGGTCCACAGGAAGTGCCAGTTCCCGCCATG | |
| | ATTGCTAGTTCAGACTATTACTTTTCTTGGCTGACTTGGGTG | |
| | ACCGACGAGCGCGTGTGCCTTCAATGGCTTAAGCGCGTCCA | |
| | AAATGTGAGCGTCCTCTCAATATGCGACTTTCGAGAAGACT | |
| | GGCAGACTTGGGATTGCCCCAAGACACAGGAGCATATTGA | |
| | AGAGAGCAGAACTGGTTGGGCCGGAGGTTTTTTCGTTTCTA | |
| | CACCCGTTTTCTCATACGACGCCATTAGCTACTACAAGATC | |
| | TTCAGTGATAAAGACGGCTACAAACACATCCACTACATCA | |
| | AGGATACTGTTGAAAACGCAATTCAGATCACTAGCGGAAA | |
| | GTGGGAGGCCATCAACATTTTCAGAGTGACCCAGGACAGC | |
| | TTGTTCTACTCTTCAAATGAATTTGAGGAATATCCCGGCCG | |
| | AAGGAACATCTATCGGATCTCCATCGGCTCATATCCTCCCA | |
| | GTAAAAAGTGCGTGACTTGCCATCTTCGAAAGGAGAGATG | |
| | CCAGTACTACACTGCATCCTTTAGCGACTACGCCAAGTACT | |
| | ACGCATTGGTGTGCTACGGACCTGGAATTCCAATCTCCACC | |
| | CTGCACGACGGCCGAACCGACCAGGAGATCAAGATCCTGG | |
| | AGGAGAACAAGGAGCTGGAGAACGCACTGAAAAACATCC | |
| | AGCTGCCAAAAGAAGAAATCAAGAAACTGGAAGTGGATG | |
| | AAATTACCCTGTGGTATAAGATGATCTTGCCTCCACAGTTT | |
| | GATAGGAGCAAGAAATACCCACTCCTTATTCAAGTCTATGG | |
| | GGGACCCTGCTCTCAGTCTGTCCGGAGCGTATTTGCCGTGA | |
| | ACTGGATTTCTTACCTGGCAAGTAAGGAGGGTATGGTGATT | |
| | GCTTTGGTGGACGGAAGGGGGACAGCATTTCAAGGGGATA | |
| | AGTTGCTGTATGCAGTCTACAGGAAGCTGGGAGTCTATGA | |
| | AGTGGAAGACCAGATTACCGCCGTTCGCAAGTTCATCGAG | |
| | ATGGGCTTTATCGACGAAAAACGGATTGCTATTTGGGGTTG | |
| | GTCTTACGGCGGTTATGTGTCTAGCTTGGCTTTGGCTTCCG | |
| | GCACAGGTCTTTTCAAGTGTGGCATAGCTGTGGCCCCTGTG | |
| | AGTTCCTGGGAATATTACGCCTCCGTATATACAGAGAGATT | |
| | TATGGGGCTGCCAACAAAGGATGACAACCTCGAGCACTAT | |
| | AAGAATTCCACAGTTATGGCCAGGGCAGAATACTTTCGTA | |
| | ACGTGGACTACCTGTTGATCCATGGCACCGCTGATGATAAT | |
| | GTCCATTTCCAGAACTCCGCACAGATCGCCAAGGCTCTCGT | |
| | TAACGCCCAGGTGGACTTTCAGGCTATGTGGTACTCTGACC | |
| | AGAATCATGGGCTGAGTGGTTTGTCCACTAATCACCTGTAC | |
| | ACTCACATGACACATTTCCTGAAGCAGTGCTTCTCCCTGAG | |
| | CGAC | |
| 26 | ELVVTQEPSLTVSPGGTVTLTCRSSTGAVTTSNYANWVQQKP | Cross313/316 light |
| | GQAPRGLIGGTNKRAPGTPARFSGSLLGGKAALTLSGVQPED | chain variable region |
| | EAEYYCALWYSNLWVFGGGTKLTVL | |
| 27 | EVQLLESGGGLVQPGGSLKLSCAASGFTFNTYAMNWVRQAP | Cross313 heavy chain |
| | GKGLEWVARIRSKYNNYATYYADSVKDRFTISRDDSKNTAY | variable region |
| | LQMNNLKTEDTAVYYCVRHGNFGNSYQSWFAYWGQGTLVT | |
| | VSS | |
| 28 | EVQLLESGGGLVQPGGSLKLSCAASGFTFNTYAMNWVRQAP | Cross316 heavy chain |
| | GKGLEWVARIRSKYNNYATYYADSVKDRFTISRDDSKNTAY | variable region |
| | LQMNNLKTEDTAVYYCVRHGNFGNSYVSWFLYWGQGTLVT | |
| | VSS | |
| 29 | ELVVTQEPSLTVSPGGTVTLTCRSSTGAVTTSNYANWVQQKP | Cross325 light chain |
| | GQAPRGLIGGTNKRAPGTPARFSGSLLGGKAALTLSGVQPED | variable region |
| | EAEYYCALWYSNDWVFGGGTKLTVL | |
| 30 | EVQLLESGGGLVQPGGSLKLSCAASGFTFNTYAMNWVRQAP | Cross325 heavy chain |
| | GKGLEWVARIRSKYNNYATYYADSVKDRFTISRDDSKNTAY | variable region |
| | LQMNNLKTEDTAVYYCVRHGNFGNSYVSWFAYWGQGTLVT | |
| | VSS | |
| 31 | DIVLTQSPASLAVSPGQRATITCRASKSLSYGAYSYMHWYQQ | AHF21339 light chain |
| | KPGQPPKLLIYLASNLESGVPARFSGSGSGTDFTLTINPVEAND | variable region |
| | AANYYCQHSRELPYTFGGGTKLEIK | |
| 32 | QVQLVQSGAEVKKPGASVKVSCKASGYTFTENIIHWVRQAPG | AHF21339 heavy chain |
| | QGLEWIGWFHPGSGSIKYNEKFKDRVTMTADTSISTAYMELS | variable region |
| | RLRSDDTAVYYCARHKGTGRGAMDYWGQGTLVTVSS | |
| 33 | PKSSDKTHTCPPCPAPEAAGGPSVFLFPPKPKDTLMISRTPEVT | Amino acid sequence |
| | CVVVDVSHEDPEVKFNWYVDGVEVHNAKTKPREEQYNSTY | of first Fc region |
| | RVVSVLTVLHQDWLNGKEYKCKVSNKALPAPIEKTISKAKGQ | |
| | PREPQVYTLPPCREEMTKNQVSLWCLVKGFYPSDIAVEWESN | |
| | GQPENNYKTTPPVLDSDGSFFLYSKLTVDKSRWQQGNVFSCS | |
| | VMHEALHNHYTQKSLSLSPGK | |
| 34 | PKSCDKTHTCPPCPAPEAAGGPSVFLFPPKPKDTLMISRTPEVT | Amino acid sequence |
| | CVVVDVSHEDPEVKFNWYVDGVEVHNAKTKPREEQYNSTY | of second Fc region |
| | RVVSVLTVLHQDWLNGKEYKCKVSNKALPAPIEKTISKAKGQ | |
| | PREPQVCTLPPSREEMTKNQVSLSCAVKGFYPSDIAVEWESNG | |
| | QPENNYKTTPPVLDSDGSFFLVSKLTVDKSRWQQGNVFSCSV | |
| | MHEALHNHYTQKSLSLSPGK | |
| 35 | GGGGSGGGGSGGGGS | linker peptide 1 |
| 36 | GGGGS | linker peptide 2 |

Hereinafter, the solutions of the present disclosure will be explained in conjunction with examples. Those skilled in the art will understand that the following examples are only used to illustrate the present disclosure and should not be considered as limiting the scope of the present disclosure. Where specific techniques or conditions are not specified in the examples, they are performed according to techniques or conditions described in the literature in the art or according to the product specification. The reagents or instruments used without specifying the manufacturer are all conventional products that can be obtained commercially.

### Example 1: Production of Antibody

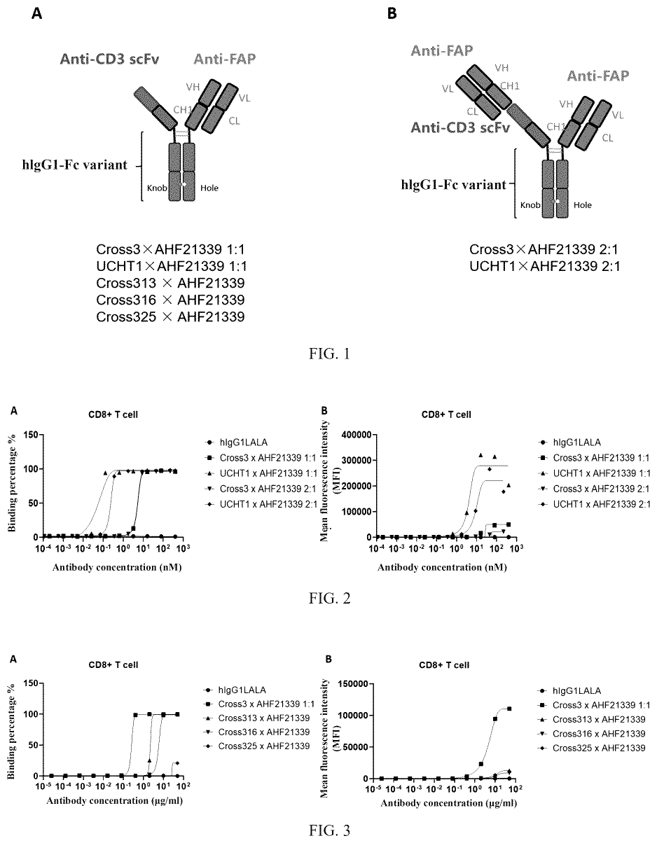

The specific experimental operations for the production of the antibody were as follows: (1) ExpiCHO cells (purchased from Thermo Fisher) were cultured using ExpiCHO Expression Medium (purchased from Thermo Fisher), and the cell concentration was adjusted to 6×10⁶/mL to obtain an ExpiCHO cell solution; (2) when the bispecific antibody was in configuration A (A in FIG. 1), pcDNA3.4 vectors containing a CD3 antibody, an FAP antibody heavy chain, and an FAP antibody light chain (synthesized by commissioned GenScript, Nanjing) were added into 2 mL of OptiSFM medium (purchased from Thermo Fisher) at a mass ratio of 1:1:1 to obtain a solution a; or when the bispecific antibody was in configuration B (B in FIG. 1), pcDNA3.4 vectors containing an FAP heavy chain-CD3 antibody, an FAP antibody heavy chain, and an FAP antibody light chain (synthesized by GenScript, Nanjing) were added into 2 mL of OptiSFM medium (purchased from Thermo Fisher) at a ratio of 1:1:1 to obtain solution a; (3) 160 μL of ExpiFectamineCHO transfection reagent (purchased from Thermo Fisher) was added to 2 mL of OptiSFM medium (purchased from Thermo Fisher) to obtain a solution b; (4) then the solution a and the solution b were mixed to obtain a transfection mixture, and the entire transfection mixture was added to 50 mL of the ExpiCHO cell solution within 5 minutes; (5) the cells were cultured under conditions of 37 °C and 5% CO₂ for 1 day, 8 mL of Feed and 300 μL of Enhancer (purchased from Thermo Fisher) were added into the solution, cultured under conditions of 32 °C and 5% CO₂ for 9 days and the culture supernatant was harvested, with 8 mL of Feed added on Day 5; and (6) affinity purification was performed on the culture supernatant by utilizing a Protein A purification column (purchased from Nano-micro) to obtain the target antibody.

In this example, a total of seven CD3 and FAP bispecific antibodies in two configurations were prepared to investigate various properties of the recombinant antibodies of the present disclosure. Specific information of the seven bispecific antibodies is shown in Table 2. The configurations of the antibodies A and B are shown in FIG. 1.

TABLE 2

| | Antibody | First antigen- | Second antigen- |
|---|---|---|---|
| Antibody name | configuration | binding region | binding region |
| Cross3 × AHF21339 1:1 | A | SEQ ID NO: 18 | SEQ ID NO: 19 |
| | | | SEQ ID NO: 20 |
| Cross3 × AHF21339 2:1 | B | SEQ ID NO: 21 | SEQ ID NO: 19 |
| | | SEQ ID NO: 20 | SEQ ID NO: 20 |
| UCHT1 × AHF21339 1:1 | A | SEQ ID NO: 22 | SEQ ID NO: 19 |
| | | | SEQ ID NO: 20 |
| UCHT1 × AHF21339 2:1 | B | SEQ ID NO: 23 | SEQ ID NO: 19 |
| | | SEQ ID NO: 20 | SEQ ID NO: 20 |
| Cross313 × AHF21339 | A | SEQ ID NO: 15 | SEQ ID NO: 19 |
| | | | SEQ ID NO: 20 |
| Cross316 × AHF21339 | A | SEQ ID NO: 16 | SEQ ID NO: 19 |
| | | | SEQ ID NO: 20 |
| Cross325 × AHF21339 | A | SEQ ID NO: 17 | SEQ ID NO: 19 |
| | | | SEQ ID NO: 20 |

The seven bispecific antibodies were as follows: Cross3×AHF21339 1:1 (the amino acid sequence of scFv+Fc of CD3 is as set forth in SEQ ID NO: 18, the amino acid sequence of the FAP antibody heavy chain is as set forth in SEQ ID NO: 19, and the amino acid sequence of the FAP antibody light chain is as set forth in SEQ ID NO: 20); Cross3×AHF21339 2:1 (the amino acid sequence of the heavy chain variable region+CH1 region of the FAP antibody and scFv+Fc of the CD3 antibody is as set forth in SEQ ID NO: 21, the amino acid sequence of the FAP antibody heavy chain is as set forth in SEQ ID NO: 19, and the amino acid sequence of the FAP antibody light chain is as set forth in SEQ ID NO: 20), UCHT1×AHF21339 1:1 (the amino acid sequence of scFv+Fc of the CD3 antibody is as set forth in SEQ ID NO: 22, the amino acid sequence of the FAP antibody heavy chain is as set forth in SEQ ID NO: 19, and the amino acid sequence of the FAP antibody light chain is as set forth in SEQ ID NO: 20); UCHT1×AHF21339 2:1 (the amino acid sequence of the heavy chain variable region+CH1 region of the FAP antibody and scFv+Fc of the CD3 antibody is as set forth in SEQ ID NO: 23, the amino acid sequence of the FAP antibody heavy chain is as set forth in SEQ ID NO: 19, and the amino acid sequence of the FAP antibody light chain is as set forth in SEQ ID NO: 20); Cross313×AHF21339 (the amino acid sequence of scFv+Fc of the CD3 antibody is as set forth in SEQ ID NO: 15, the amino acid sequence of the FAP antibody heavy chain is as set forth in SEQ ID NO: 19, and the amino acid sequence of the FAP antibody light chain is as set forth in SEQ ID NO: 20); Cross316×AHF21339 (the amino acid sequence of scFv+Fc of the CD3 antibody is as set forth in SEQ ID NO: 16, the amino acid sequence of the FAP antibody heavy chain is as set forth in SEQ ID NO: 19, and the amino acid sequence of the FAP antibody light chain is as set forth in SEQ ID NO: 20); and Cross325×AHF21339 (the amino acid sequence of scFv+Fc of the CD3 antibody is as set forth in SEQ ID NO: 17, the amino acid sequence of the FAP antibody heavy chain is as set forth in SEQ ID NO: 19, and the amino acid sequence of the FAP antibody light chain is as set forth in SEQ ID NO: 20).

### Example 2: Flow Cytometry Binding Assay of the Antibody

Flow cytometry assay was used to detect the binding properties of the bispecific antibody. The bispecific antibody was added to cells, and the intensity of the signal after the addition of the antibody was used to determine the binding properties of the antibody and the cell.

(1) PBMCs were diluted to a density of 2×10⁶/ml with PBS and added into a 1.5 ml Eppendorf (EP) tube at a volume of 100 μl/tube. 10 μl/tube of goat serum was added to each tube, and incubated at 4 °C for 30 minutes for blocking. The serially diluted bispecific antibodies Cross3×AHF21339 1:1, Cross3×AHF21339 2:1, UCHT1×AHF21339 1:1, UCHT1×AHF21339 2:1, Cross313×AHF21339, Cross316×AHF21339, and Cross325×AHF21339 and control hIgG1LALA (purchased from Biointron) were added, and the cells were incubated at 4 °C for 30 minutes. 1 ml of PBS was added into the EP tube, and the EP tube was centrifuged at 3,500 rpm for 5 minutes at 4 °C. The supernatant was discarded, and the cells were washed again with PBS. After centrifugation, the supernatant was discarded, and the cells were resuspended with 100 μl/tube of PBS. 1 μl/tube of Alexa-647-labeled goat anti-human IgG secondary antibody (purchased from Jackson Lab) and 0.5 μl/tube of PerCP-Cy5.5-labeled goat anti-human CD8 antibody were added, and incubated at 4 °C in the dark for 30 minutes. The cells were washed twice with PBS, and the supernatant was discarded after centrifugation. The cells were resuspended with 200 μl/tube of PBS and detected using a flow cytometry.

The binding intensities and binding activities of the seven bispecific antibodies to CD8 T cells are shown in FIG. 2 and FIG. 3. FIG. 2 shows that Cross3×AHF21339 1:1, Cross3×AHF21339 2:1, UCHT1×AHF21339 1:1, and UCHT1×AHF21339 2:1 antibodies can bind to CD8 T cells, and the bispecific antibodies in conformation B has weaker T cell binding capacity than those in conformation A, and the Cross3-based FAP bispecific antibody has weaker T cell binding activity than that of the UCHT1-based FAP bispecific antibody. FIG. 3 shows that Cross3×AHF21339 1:1, Cross313×AHF21339, Cross316×AHF21339, and Cross325×AHF21339 antibodies can bind to CD8 T cells, and the order of binding intensities to T cells was Cross3×AHF21339 1:1, Cross313×AHF21339, Cross316×AHF21339, Cross325×AHF21339.

(2) Preparation of A-375-FAP cells: HEK293T cells were plated into six-well plates at 5×10⁵ cells/well and cultured overnight in DMEM medium without antibiotics. Before transfection, the culture medium was discarded, and 1 ml of fresh DMEM medium without antibiotics was added. pLVX-EF1a-FAP-IRES-puro (synthesized by commissioned GenScript, Nanjing, a coding sequence (SEQ ID NO: 25) of the human FAP protein (SEQ ID NO: 24) was inserted between restriction sites EcoRI and BamHI of the pLVX-EF1a-IRES-puro vector (purchased from Unibio)), pMD2G (purchased from Unibio), and psPAX2 (purchased from Unibio) vectors (3 μg in total) were added to 200 μl of serum-free DMEM medium at a ratio of 2:1:1, followed by an addition of 12 μg of polyetherimide (PEI, purchased from Polysciences). After uniformly mixing, the mixture was left to stand for 16 minutes, and then all the liquid was added into a six-well plate containing HEK293T cells. After 6 hours of culture, the culture medium was removed, and fresh complete DMEM medium was added for cultivation. 48 hours after transfection, the cell culture supernatant was collected and filtered through a 0.45 μm filter (purchased from Millipore) to obtain the viral supernatant. The entire viral supernatant was added to a 6-well plate containing 1×10⁴ A-375 cells, and polybrene (purchased from Sigma) at a final concentration of 4 μg/ml was added and cultured for 12 hours. The supernatant was then removed, and fresh complete DMEM medium was added. The obtained cells were the A-375-FAP cells.

A-375-FAP tumor cells were diluted to a density of 2×10⁶/ml with PBS and added into a 1.5 ml EP tube at a volume of 100 μl/tube. 10 μl/tube of goat serum was added to each tube, and incubated at 4 °C for 30 minutes for blocking. The serially diluted bispecific antibodies Cross3×AHF21339 1:1, Cross3×AHF21339 2:1, UCHT1×AHF21339 1:1, UCHT1×AHF21339 2:1, Cross313×AHF21339, Cross316×AHF21339, and Cross325×AHF21339, and control hIgG1LALA (purchased from Biointron) were added, and the cells were incubated at 4 °C for 30 minutes. 1 ml of PBS was added to the EP tube, and the EP tube was centrifuged at 3,500 rpm for 5 minutes at 4 °C. The supernatant was discarded, and the cells were washed again with PBS. After centrifugation, the supernatant was discarded, and the cells were resuspended with 100 μl/tube of PBS. 1 μl/tube of Alexa-647-labeled goat anti-human IgG secondary antibody (purchased from Jackson Lab) was added, and incubated at 4 °C in the dark for 30 minutes. The cells were washed twice with PBS, and the supernatant was discarded after centrifugation. The cells were resuspended with 200 μl/tube of PBS and detected using a flow cytometry.

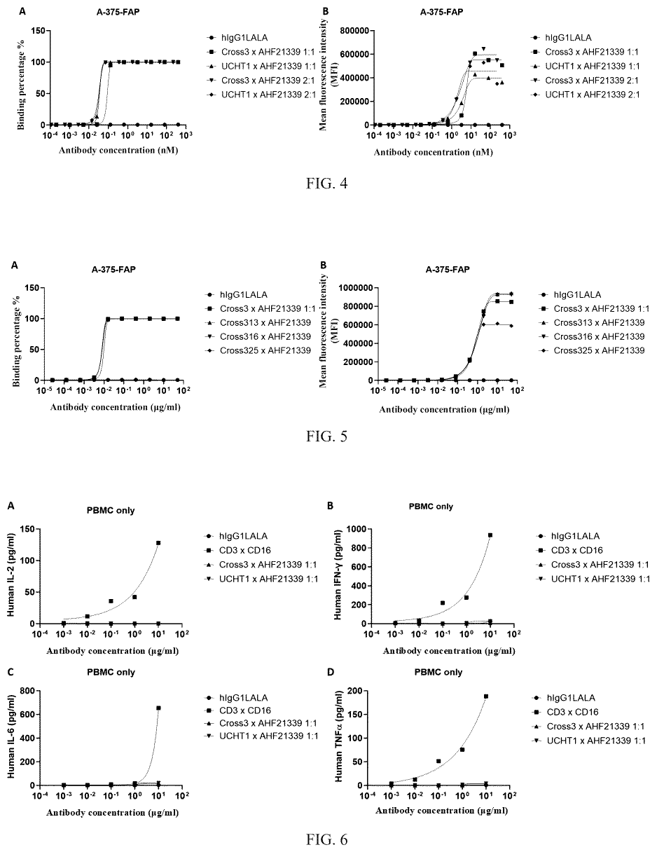

The binding intensities and binding activities of the seven bispecific antibodies to A-375-FAP cells are shown in FIG. 4 and FIG. 5. FIG. 4 shows that Cross3×AHF21339 1:1, Cross3×AHF21339 2:1, UCHT1×AHF21339 1:1, and UCHT1×AHF21339 2:1 antibodies can all bind to A-375-FAP cells, and the activity (EC50) of the bispecific antibody in B configuration in binding to FAP is lower than that of the bispecific antibody in A configuration. FIG. 5 shows that antibodies Cross3×AHF21339 1:1, Cross313×AHF21339, Cross316×AHF21339, and Cross325×AHF21339 can all bind to A-375-FAP cells, and the activities (EC50) of all the bispecific antibodies were comparable.

### Example 3: Assay on Promotion of Cytokine Secretion from PBMCs by Bispecific Antibodies

(1) Experiment on Promotion of Cytokine Secretion from PBMCs by Bispecific Antibodies in the Absence of Target Protein (without Tumor Cells)

The bispecific antibodies were added to the PBMC culture system. After culturing for 48 hours, the culture supernatant was collected, and the cytokine content in the supernatant was detected, to evaluate cytokine release properties induced by the bispecific antibodies in the absence of target protein.

(a) The bispecific antibodies Cross3×AHF21339 1:1, UCHT1×AHF21339 1:1, Cross313×AHF21339, Cross316×AHF21339, Cross325×AHF21339, and control hIgG1LALA (purchased from Biointron) were serially diluted with the complete RPMI 1640 medium and added into a 96-well plate at 100 μl/well.

(b) PBMCs (purchased from Shanghai Saily Biotechnology Co., Ltd.) were diluted to a density of 1×10⁶/ml with the complete RPMI 1640 medium and added into the 96-well plate at 100 μl/well, and the 96-well plate was cultured in a 37 °C, 5% CO₂ incubator for 48 hours.

(c) The plate was centrifuged at 300 g for 10 minutes at a room temperature, and the cell culture supernatant was collected.

(d) The cytokine content in the supernatant was detected using a CBA kit (purchased from BD).

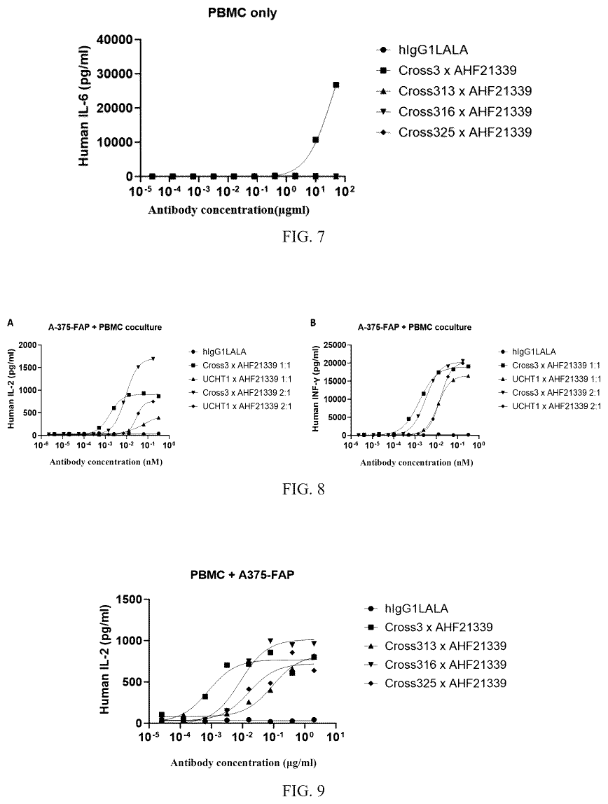

The results are shown in FIG. 6 and FIG. 7. The PBMCs used in each experimental group in FIG. 6 were from the same donor. The PBMCs used in each experimental group in FIG. 7 were from the same donor. The PBMCs used in the experiments of FIG. 6 and FIG. 7 were from different donors. FIG. 6 shows that the antibodies Cross3×AHF21339 1:1 and UCHT1×AHF21339 1:1 did not cause spontaneous activation of PBMCs in the absence of the target protein (FAP), demonstrating certain safety. FIG. 7 shows that the antibodies Cross313×AHF21339, Cross316×AHF21339, and Cross325×AHF21339 did not cause spontaneous activation of PBMCs in the absence of the target protein (FAP), and Cross3×AHF21339 1:1 induced the release of the pro-inflammatory cytokine IL-6 at high concentrations, indicating that the safety of Cross313×AHF21339, Cross316×AHF21339, and Cross325×AHF21339 was higher than that of Cross3×AHF21339 1:1.

(2) Experiment on Promotion of Cytokine Secretion from PBMCs by Bispecific Antibodies in the Presence of Target Protein (with Tumor Cells)

Bispecific antibodies were added to the co-incubation system of PBMCs and A-375-FAP melanoma cells. After culturing for 48 hours, the culture supernatant was collected, and the cytokine content in the supernatant was detected, to evaluate cytokine release properties induced by the bispecific antibodies.

(a) A-375-FAP cells were diluted to a density of 1×10⁵/ml with the complete RPMI 1640 medium, added into a 96-well plate, and cultured in a 37 °C, 5% CO₂ incubator for 24 hours.

(b) The bispecific antibodies Cross3×AHF21339 1:1, Cross3×AHF21339 2:1, UCHT1×AHF21339 1:1, UCHT1×AHF21339 2:1, Cross313×AHF21339, Cross316×AHF21339, and Cross325×AHF21339, and control hIgG1LALA (purchased from Biointron) were serially diluted with the complete RPMI 1640 medium and added into the 96-well plate at 20 μl/well.

(c) PBMCs (purchased from Shanghai Saily Biotechnology Co., Ltd.) were diluted to a density of 1.25×10⁶/ml with the complete RPMI 1640 medium and added into the 96-well plate at 80 μl/well.

(d) The 96-well plate was cultured in a 37 °C, 5% CO₂ incubator for 48 hours.

(e) The plate was centrifuged at 300 g for 10 minutes at a room temperature, and the cell culture supernatant was collected.

(f) The cytokine content in the supernatant was detected using a CBA kit (purchased from BD).

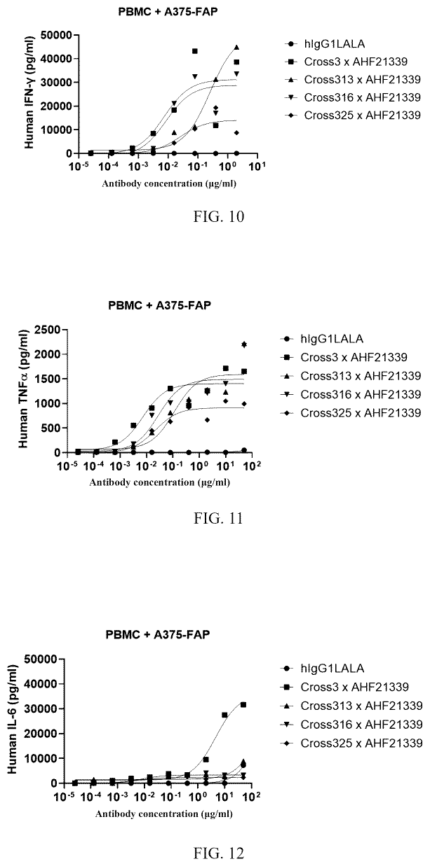

The results are shown in FIG. 8 to FIG. 12. FIG. 8 shows that the antibodies Cross3×AHF21339 1:1, Cross3×AHF21339 2:1, UCHT1×AHF21339 1:1, and UCHT1×AHF21339 2:1 can activate PBMCs and promote the secretion of cytokines IL-2, IFN-γ, IL-6, and TNF-α in the presence of the target protein (FAP), and the activity of the Cross3 bispecific antibodies was superior to that of the UCHT1 bispecific antibodies, with Cross3×AHF21339 1:1 having the optimal activity. FIG. 9 shows that the antibodies Cross3×AHF21339 1:1, Cross313×AHF21339, Cross316×AHF21339, and Cross325×AHF21339 can activate PBMCs and secrete IL-2 in the presence of the target protein (FAP). FIG. 10 shows that the antibodies Cross3×AHF21339 1:1, Cross313×AHF21339, Cross316×AHF21339, and Cross325×AHF21339 can activate PBMCs and secrete IFN-γ in the presence of the target protein (FAP). FIG. 11 shows that the antibodies Cross3×AHF21339 1:1, Cross313×AHF21339, Cross316×AHF21339, and Cross325×AHF21339 can activate PBMCs and secrete TNFα in the presence of the target protein (FAP). FIG. 12 shows that the antibodies Cross3×AHF21339 1:1, Cross313×AHF21339, Cross316×AHF21339, and Cross325×AHF21339 can activate PBMCs and secrete the pro-inflammatory cytokine IL-6 in the presence of the target protein (FAP).

In summary, the antibodies Cross313×AHF21339, Cross316×AHF21339, and Cross325×AHF21339 can maintain a high ability to kill tumor cells while demonstrating high safety.

### Example 4: Assay on Promotion of T Cell Proliferation by Bispecific Antibodies

PBMC cells were labeled with CFSE fluorochrome. The bispecific antibodies were added to the co-incubation system of PBMCs and A-375-FAP cells. After culturing for 72 h, the CFSE fluorescence intensity in CD4 T cells and CD8 T cells was detected by flow cytometry, to evaluate T cell proliferation properties induced by the bispecific antibodies.

(1) A-375-FAP cells were diluted to a density of 1×10⁵/ml with the complete RPMI 1640 medium and added into a 96-well plate.

(2) The bispecific antibodies Cross3×AHF21339 1:1, Cross3×AHF21339 2:1, UCHT1×AHF21339 1:1, UCHT1×AHF21339 2:1, Cross313×AHF21339, Cross316×AHF21339, and Cross325×AHF21339, and control hIgG1LALA (purchased from Biointron) were serially diluted with the complete RPMI 1640 medium and added into the 96-well plate at 20 μl/well.

(3) PBMCs were labeled with 5 μM of CFSE. After labeling, PBMCs (purchased from Shanghai Saily Biotechnology Co., Ltd.) were diluted to a density of 1.25×10⁶/ml with the complete RPMI 1640 medium and added into the 96-well plate at 80 μl/well.

(4) The 96-well plate was cultured in a 37 °C, 5% CO₂ incubator for 72 hours.

(5) PerCP-Cy5.5-labeled CD8 antibody and BV605-labeled CD4 antibody were added, and incubated at 4 °C in the dark for 30 minutes.

(6) The cells were washed twice with PBS, and the supernatant was discarded after centrifugation.

(7) The cells were resuspended with 200 μl/tube of PBS and detected using a flow cytometry.

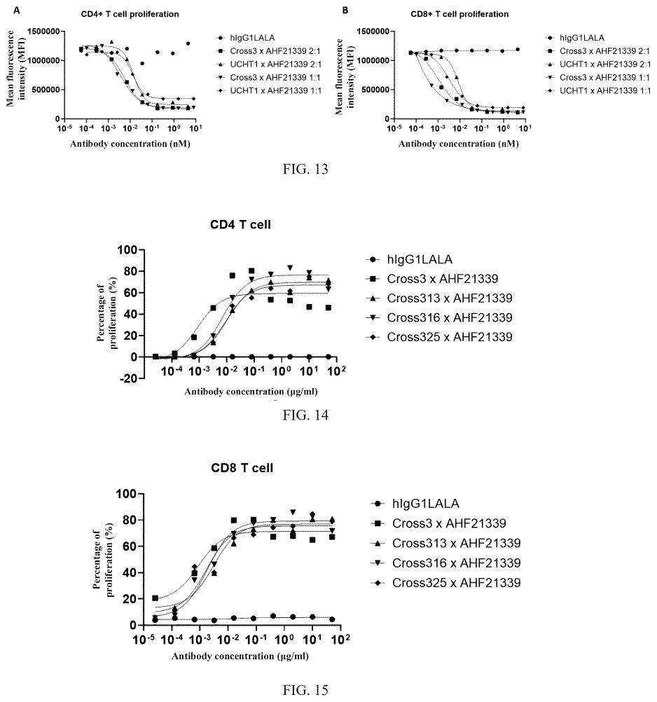

The results are shown in FIG. 13 to FIG. 15. FIG. 13 shows that the antibodies Cross3×AHF21339 1:1, Cross3×AHF21339 2:1, UCHT1×AHF21339 1:1, and UCHT1×AHF21339 2:1 can promote the activation of CD4 and CD8 T cell, and the activity of Cross3 bispecific antibodies was superior to that of UCHT1 bispecific antibodies, with Cross3×AHF21339 1:1 having the optimal activity. FIG. 14 shows that the antibodies Cross3×AHF21339 1:1, Cross313×AHF21339, Cross316×AHF21339, and Cross325×AHF21339 can all promote the proliferation of CD4 T cells. FIG. 15 shows that the antibodies Cross3×AHF21339 1:1, Cross313×AHF21339, Cross316×AHF21339, and Cross325×AHF21339 can promote the proliferation of CD8 T cells.

### Example 5: Assay on Promotion of PBMC-Mediated Killing of FAP-Positive Cells by Bispecific Antibodies

The capability of the bispecific antibody to promote PBMC-mediated killing of A-375-FAP human melanoma cells was detected.

(a) Complete RPMI-1640 medium was added into a 16-well RTCA plate at a volume of 50 μl/well for plate calibration.

(b) A-375-FAP tumor cells were diluted to a density of 2×10⁵/mL with the complete RPMI-1640 medium, and added individually into the RTCA plate obtained in the step (1) at a volume of 50 μl/well. Then, the cell index was monitored for 24 hours under conditions of 37 °C. and 5% CO₂ using an instrument xCELLigence RTCA MP.

(c) The antibodies Cross3×AHF21339 2:1, 1:1, Cross3×AHF21339 UCHT1×AHF21339 1:1, UCHT1×AHF21339 2:1, Cross313×AHF21339, Cross316×AHF21339, and Cross325×AHF21339 and control hIgG1LALA (purchased from Biointron), which were serially diluted bispecific with the complete RPMI-1640 medium, were added into the RTCA plate obtained in the step (b), with an addition volume of 20 μl/well.

(d) PBMCs (purchased from Shanghai Saily Biotechnology Co., Ltd.) were diluted to a density of 1.25×10⁶/ml with the complete RPMI-1640 medium, and added into the RTCA plate obtained in the step (c) at a volume of 80 μl/well.

(e) The reaction system obtained in the step (d) was placed under conditions of 37 °C and 5% CO₂, and the cell index was monitored for 24 hours using the instrument xCELLigence RTCA MP.

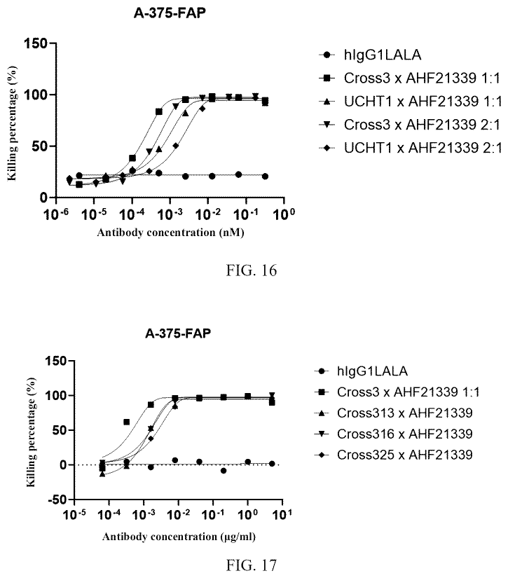

The results are shown in FIG. 16. FIG. 16 shows that the antibodies Cross3×AHF21339 1:1, Cross3×AHF21339 2:1, UCHT1×AHF21339 1:1, and UCHT1×AHF21339 2:1 can promote PBMC-mediated killing of A-375-FAP cells, the pro-killing activity of the bispecific antibody in 1:1 configuration was stronger than that of the bispecific antibody in 2:1 configuration, the Cross3 bispecific antibodies were superior to the UCHT1 bispecific antibodies, and Cross3×AHF21339 1:1 had the highest pro-killing activity. The results are shown in FIG. 17. FIG. 17 shows that the antibodies Cross3×AHF21339 1:1, Cross313×AHF21339, Cross316×AHF21339, and Cross325×AHF21339 can all promote PBMC-mediated killing of A-375-FAP cells, and the antibodies Cross313×AHF21339, Cross316×AHF21339, and Cross325×AHF21339 exhibit comparable pro-killing activity, which was lower than that of Cross3×AHF21339 1:1, but can all promote complete killing.

### Example 6: ELISA Binding Assay of the Antibody of the Present Disclosure

ELISA assay was used to detect the binding properties of the bispecific antibody. The antigen protein was coated into a 96-well plate, and the intensity of the signal after the addition of the antibody was used to determine the binding properties of the bispecific antibody and the antigen protein.

(1) FAP-His protein (purchased from Acro) was diluted to a concentration of 2 μg/ml with PBS buffer and added to a 96-well plate at a volume of 100 μl/well. The plate was placed at 4 °C overnight. The PBS buffer in the 96-well plate was aspirated. The plate was washed 6 times with PBST (PBS containing 0.1% Tween 20, pH7.2) buffer, and then 200 μl/well of PBS/10% BSA was added. The plate was incubated at 37 °C for 2 hours for blocking. The blocking solution was removed, and the plate was washed 6 times with PBST. The to-be-tested bispecific antibodies Cross3×AHF21339 1:1, Cross313×AHF21339, Cross316×AHF21339, and Cross325×AHF21339, and control hIgG1LALA (purchased from Biointron), which were serially diluted with PBST/0.05% BSA, were added at a volume of 100 μl/well. The plate was incubated at 37 °C for 1 hour. The reaction system was removed, and the plate was washed 6 times with PBST, and then HRP (horseradish peroxidase)-labeled anti-human IgG secondary antibody (purchased from Jacksonlab) diluted with PBST/0.05% BSA was added at 100 μl/well. The plate was incubated at 37 °C for 1 hour. After the plate was washed 6 times with PBST, 80 μl/well of TMB (tetramethylbenzidine) was added, and the plate was incubated at room temperature for 3 minutes. 80 μl/well of 4M sulfuric acid was added to terminate the reaction. The absorbance value was read at 450 nm using an enzyme reader.

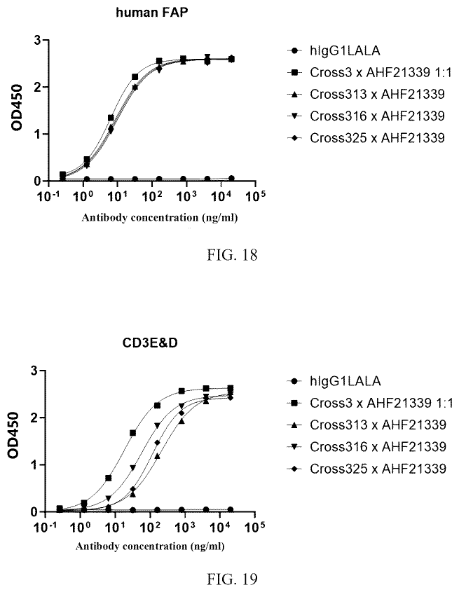

The results are shown in FIG. 18. FIG. 18 shows that the antibodies Cross3×AHF21339 1:1, Cross313×AHF21339, Cross316×AHF21339, and Cross325×AHF21339 can all bind to the FAP protein, with comparable binding capabilities.

(2) CD3E&D protein (purchased from Acro) was diluted to a concentration of 2 μg/ml with PBS buffer and added to a 96-well plate at a volume of 100 μl/well. The plate was placed at 4 °C overnight. The PBS buffer in the 96-well plate was aspirated. The plate was washed 6 times with PBST (PBS containing 0.1% Tween 20, pH7.2) buffer, and then 200 μl/well of PBS/10% BSA was added. The plate was incubated at 37 °C for 2 hours for blocking. The blocking solution was removed, and the plate was washed 6 times with PBST. The to-be-tested bispecific antibodies Cross3×AHF21339 1:1, Cross313×AHF21339, Cross316×AHF21339, and Cross325×AHF21339, and control hIgG1LALA (purchased from Biointron), which were serially diluted with PBST/0.05% BSA, were added at a volume of 100 μl/well. The plate was incubated at 37 °C for 1 hour. The reaction system was removed, and the plate was washed 6 times with PBST, and then HRP (horseradish peroxidase)-labeled anti-human IgG secondary antibody (purchased from Southern biotech) diluted with PBST/0.05% BSA was added at 100 μl/well. The plate was incubated at 37 °C for 1 hour. After the plate was washed 6 times with PBST, 80 μl/well of TMB (tetramethylbenzidine) was added, and the plate was incubated at room temperature for 3 minutes. 80 μl/well of 4M sulfuric acid was added to terminate the reaction. The absorbance value was read at 450 nm using an enzyme reader.

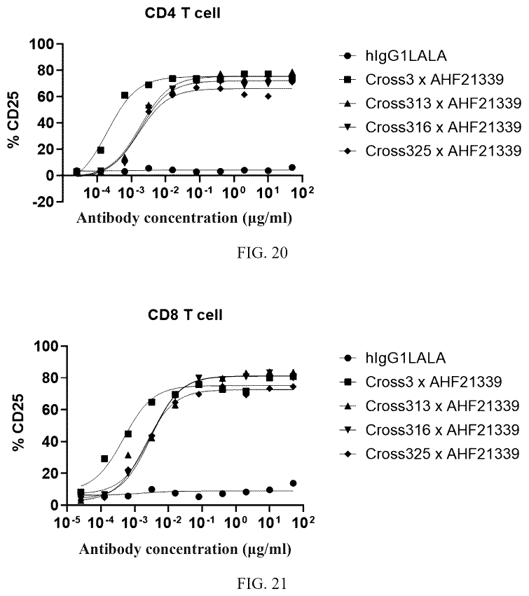

The results are shown in FIG. 19. FIG. 19 shows that the antibodies Cross3×AHF21339 1:1, Cross313×AHF21339, Cross316×AHF21339, and Cross325×AHF21339 of the present disclosure can all bind to the CD3E&D protein, and the activities of Cross313×AHF21339, Cross316×AHF21339, and Cross325×AHF21339 antibodies to bind to CD3 was weaker than that of Cross3×AHF21339 1:1.

### Example 7: Assay on Promotion of T Cell Activation by Bispecific Antibody

Bispecific antibodies were added to the co-incubation system of PBMCs and A-375-FAP cells. After 48 hours of culture, the expression of activation markers CD25 and CD69 on the surface of CD4 T cells and CD8 T cells was detected using a flow cytometry, to evaluate the T cell activation properties induced by the bispecific antibodies.

(1) A-375-FAP cells were diluted to a density of 1×10⁵/ml with the complete RPMI 1640 medium and added into a 96-well plate.

(2) The bispecific antibodies Cross3×AHF21339 1:1, Cross313×AHF21339, Cross316×AHF21339, and Cross325×AHF21339, and control hIgG1LALA (purchased from Biointron) were serially diluted with the complete RPMI 1640 medium and added into the 96-well plate at 20 μl/well.

(3) PBMCs (purchased from Shanghai Saily Biotechnology Co., Ltd.) were diluted to a density of 1.25×10⁶/ml with the complete RPMI 1640 medium and added into the 96-well plate at 80 μl/well.

(4) The 96-well plate was cultured in a 37 °C, 5% CO₂ incubator for 48 hours.

(5) CD4, CD8, CD25, and CD69 antibodies were added, and incubated at 4 °C in the dark for 30 minutes.

(6) The cells were washed twice with PBS, and the supernatant was discarded after centrifugation.

(7) The cells were resuspended with 200 μl/tube of PBS and detected using a flow cytometry.

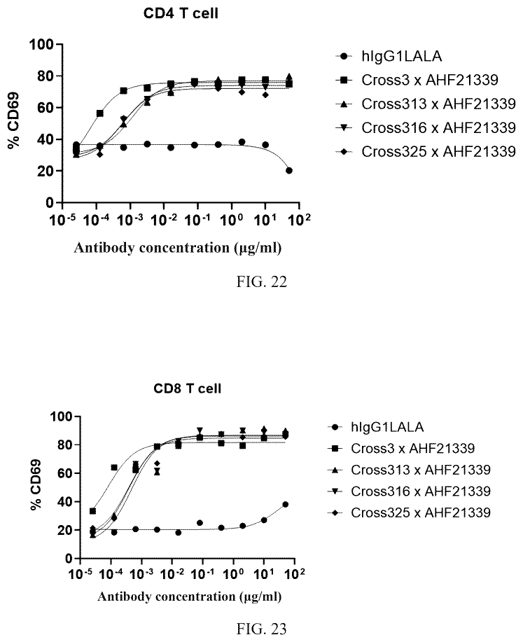

The results are shown in FIG. 20 to FIG. 23. FIG. 20 shows that the antibodies Cross3×AHF21339 1:1, Cross313×AHF21339, Cross316×AHF21339, and Cross325×AHF21339 can all promote the activation of CD4 T cells and express CD25. FIG. 21 shows that the antibodies Cross3×AHF21339 1:1, Cross313×AHF21339, Cross316×AHF21339, and Cross325×AHF21339 can all promote the activation of CD8 T cells and express CD25. FIG. 22 shows that the antibodies Cross3×AHF21339 1:1, Cross313×AHF21339, Cross316×AHF21339, and Cross325×AHF21339 can all promote the activation of CD4 T cells and express CD69. FIG. 23 shows that the antibodies Cross3×AHF21339 1:1, Cross313×AHF21339, Cross316×AHF21339, and Cross325×AHF21339 can all promote the activation of CD8 T cells and express CD69.

The above experimental results show that the bispecific antibodies of the present disclosure can bind to CD3 and FAP, which promotes T cell activation and cytokine secretion, effectively promoting killing of tumor cells by PBMCs, and demonstrating satisfactory anticancer activity. In addition, the bispecific antibodies of the present disclosure can achieve high-affinity binding to FAP and low-affinity binding to CD3, and have higher anticancer activity. In summary, the bispecific antibodies of the present disclosure can promote immune cells to fight cancer, have satisfactory anticancer activity and higher safety, and possess satisfactory clinical application value and drug development value.

In the description of the present specification, the descriptions of the reference terms such as “one embodiment”, “some embodiments”, “an example”, “a specific example”, “some implementations”, and “some examples” mean that the specific features, structures, materials or characteristics described in combination with the embodiments or examples are included in at least one embodiment or example of the present disclosure. In the present specification, the schematic expressions of the above terms do not necessarily refer to the same embodiment or example. Moreover, the specific features, structures, materials or characteristics described herein may be combined in any one or more embodiments or examples in an appropriate mode. In addition, without mutual contradiction, different embodiments or examples and features of the different embodiments or examples described in the present specification may be combined and integrated by those skilled in the art.

Although the embodiments of the present disclosure have been illustrated and described above, it can be understood that the above embodiments are illustrative and should not be construed as limiting the present disclosure. Those skilled in the art can make changes, modifications, substitutions, and variations to the above embodiments within the scope of the present disclosure.

What is claimed is:

1. A recombinant antibody, at least comprising:
a first binding region, wherein the first binding region comprises an antibody or antigen-binding fragment that specifically recognizes CD3; and
a second binding region, wherein the second binding region comprises an antibody or antigen-binding fragment that specifically recognizes FAP, wherein:
the antibody or antigen-binding fragment that specifically recognizes the CD3 in the first binding region comprises an scFV region and a first Fc region; and
the antibody or antigen-binding fragment that specifically recognizes the FAP in the second binding region comprises a Fab region and a second Fc region.

2. The recombinant antibody according to claim 1, wherein in the first binding region, a C-terminus of the scFV region is linked to an N-terminus of the first Fc region;
optionally, a C-terminus of a heavy chain variable region of the scFV region is linked to an N-terminus of a light chain variable region via a linker peptide 1, and a C-terminus of the light chain variable region is linked to the N-terminus of the first Fc region via a linker peptide 2.

3. The recombinant antibody according to claim 1, wherein:
the antibody or antigen-binding fragment that specifically recognizes the FAP comprises light chain variable region CDR1, CDR2, CDR3 sequences as set forth in SEQ ID NO: 12, SEQ ID NO: 13, and SEQ ID NO: 14, respectively, and heavy chain variable region CDR1, CDR2, CDR3 sequences as set forth in SEQ ID NO:9, SEQ ID NO: 10, and SEQ ID NO: 11, respectively;
optionally, the antibody or antigen-binding fragment that specifically recognizes the FAP has a light chain variable region as set forth in SEQ ID NO: 31 or an amino acid sequence having at least 80% identity to a sequence set forth in SEQ ID NO: 31, and a heavy chain variable region as set forth in SEQ ID NO: 32 or an amino acid sequence having at least 80% identity to a sequence set forth in SEQ ID NO: 32;
optionally, the antibody or antigen-binding fragment that specifically recognizes the FAP has a light chain as set forth in SEQ ID NO: 20 or an amino acid sequence having at least 80% identity to a sequence set forth in SEQ ID NO: 20, and a heavy chain as set forth in SEQ ID NO: 19 or an amino acid sequence having at least 80% identity to a sequence set forth in SEQ ID NO: 19.

4. The recombinant antibody according to claim 1, wherein the antibody or antigen-binding fragment that specifically recognizes the CD3 comprises:
light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 3, respectively;
light chain variable region CDR1, CDR2, CDR3 sequences as set forth in SEQ ID NO: 4, GTN, and SEQ ID NO: 5, respectively, and heavy chain variable region CDR1, CDR2, CDR3 sequences as set forth in SEQ ID NO: 1, SEQ ID NO: 2, and SEQ ID NO: 6, respectively; or
light chain variable region CDR1, CDR2, CDR3 sequences as set forth in SEQ ID NO: 4, GTN, and SEQ ID NO:8, respectively, and heavy chain variable region CDR1, CDR2, CDR3 sequences as set forth in SEQ ID NO: 1, SEQ ID NO: 2, and SEQ ID NO: 7, respectively.

5. The recombinant antibody according to claim 1, wherein the antibody or antigen-binding fragment that specifically recognizes the CD3 comprises any one of:
(a) a light chain variable region as set forth in SEQ ID NO: 26 and a heavy chain variable region as set forth in SEQ ID NO: 27;
(b) a light chain variable region as set forth in SEQ ID NO: 26 and a heavy chain variable region as set forth in SEQ ID NO: 28;
(c) a light chain variable region as set forth in SEQ ID NO: 29 and a heavy chain variable region as set forth in SEQ ID NO: 30; or
amino acid sequences having at least 80% sequence identity to any one of (a), (b), and (c).

6. The recombinant antibody according to claim 1, wherein the first Fc region is linked to the second Fc region via a knob-into-hole structure;
optionally, at least part of each of the first Fc region and the second Fc region is from at least one of a murine antibody, a primate-derived antibody, or a mutant thereof;
optionally, at least part of each of the first Fc region and the second Fc region is from human IgG1 or a mutant thereof;
optionally, the first Fc region has an amino acid sequence as set forth in SEQ ID NO: 33, and/or the second Fc region has an amino acid sequence as set forth in SEQ ID NO: 34.

7. The recombinant antibody according to claim 2, wherein the linker peptide 1 and the linker peptide 2 have each independently an amino acid sequence (GGGGS)n, where n is an integer greater than or equal to 1, and preferably n is 1, 2, 3, 4, 5, 6, 7, 8, 9, or 10;
optionally, the linker peptide 1 has an amino acid sequence as set forth in SEQ ID NO: 35;
optionally, the linker peptide 2 has an amino acid sequence as set forth in SEQ ID NO: 36.

8. The recombinant antibody according to claim 1, wherein the first binding region comprises:
an amino acid sequence as set forth in any one of SEQ ID NO: 15, SEQ ID NO: 16, or SEQ ID NO: 17; or
an amino acid sequence having at least 80% identity to SEQ ID NO: 15, SEQ ID NO: 16, or SEQ ID NO: 17.

9. The recombinant antibody according to claim 1, comprising any one of:
(d) amino acid sequences as set forth in SEQ ID NO: 15, SEQ ID NO: 19, and SEQ ID NO: 20;
(e) amino acid sequences as set forth in SEQ ID NO: 16, SEQ ID NO: 19, and SEQ ID NO: 20;
(f) amino acid sequences as set forth in SEQ ID NO: 17, SEQ ID NO: 19, and SEQ ID NO: 20; or
amino acid sequences having at least 80% sequence identity to any one of (d), (e), and (f).

10. The recombinant antibody according to claim 1, further comprising a third binding region, wherein the third binding region comprises the antibody or antigen-binding fragment that specifically recognizes FAP.

11. The recombinant antibody according to claim 10, wherein the antibody or antigen-binding fragment that specifically recognizes FAP in the third binding region comprises a Fab region, a heavy chain CH1 region of the Fab region being linked to the scFV region of the antibody or antigen-binding fragment that specifically recognizes the CD3 in the first binding region.

12. The recombinant antibody according to claim 11, wherein a C-terminus of the heavy chain CH1 region of the Fab region of the antibody or antigen-binding fragment that specifically recognizes FAP in the third binding region is linked to a heavy chain variable region of the scFV region of the antibody or antigen-binding fragment that specifically recognizes the CD3 in the first binding region;
optionally, an amino acid sequence of the Fab region of the antibody or antigen-binding fragment that specifically recognizes FAP in the third binding region and an amino acid sequence of the Fab region of the antibody or antigen-binding fragment that specifically recognizes FAP in the second binding region are identical or different.

13. An isolated polynucleotide, encoding the recombinant antibody according to claim 1.

14. An expression vector, carrying the polynucleotide according to claim 13.

15. A medicament, comprising at least one of the recombinant antibody according to claim 1.

16. A kit for detecting FAP and/or CD3 in a sample, the kit comprising at least one of the recombinant antibody according to claim 1.

17. An immunoconjugate, comprising a therapeutic agent and the recombinant antibody according to claim 1 conjugated to the therapeutic agent,
optionally, the therapeutic agent comprises any one of a polypeptide, a radionuclide, and a small molecule.

18. A method for preventing and/or treating a CD3-mediated related disease and/or an FAP-mediated related disease, the method comprising:
administering to a subject a pharmaceutically acceptable amount of the recombinant antibody according to claim 1,
wherein the CD3-mediated related disease comprises an autoimmune disease;
optionally, the autoimmune disease comprises at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis;
optionally, the FAP-mediated related disease comprises a tumor, tumor-associated fibroblasts of the tumor being FAP-positive; and
optionally, a disease caused by the tumor comprises lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

19. A method for diagnosing whether a subject is suffering from a CD3-mediated related disease and/or an FAP-mediated related disease, the method comprising:
detecting a to-be-tested sample from a subject using the recombinant antibody according to claim 1,
wherein the CD3-mediated related disease comprises an autoimmune disease;
optionally, the autoimmune disease comprises at least one of: systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, ulcerative colitis, pernicious anemia with chronic atrophic gastritis, Goodpasture syndrome, pemphigus vulgaris, bullous pemphigoid, primary biliary cirrhosis, multiple cerebrospinal sclerosis, or acute idiopathic polyneuritis;
optionally, the FAP-mediated related disease comprises a tumor, tumor-associated fibroblasts of the tumor being FAP-positive; and
optionally, a disease caused by the tumor comprises lung cancer, liver cancer, ovarian cancer, cervical cancer, skin cancer, bladder cancer, colon cancer, breast cancer, glioma, kidney cancer, gastric cancer, esophageal cancer, melanoma, oral squamous cell carcinoma, or head-and-neck cancer.

20. A method for detecting CD3 and/or FAP, the method comprising:
detecting a to-be-tested sample using the recombinant antibody according to claim 1, wherein the to-be-tested sample comprises at least one of tissue, cells, blood, serum, plasma, saliva, sweat, feces, or urine.


SEQUENCE LISTING (36 sequences)

SEQ ID NO: 1 - protein, 8 aa, synthetic construct
GFTFNTYA

SEQ ID NO: 2 - protein, 10 aa, synthetic construct
IRSKYNNYAT

SEQ ID NO: 3 - protein, 16 aa, synthetic construct
VRHGNFGNSYQSWFAY

SEQ ID NO: 4 - protein, 9 aa, synthetic construct
TGAVTTSNY

SEQ ID NO: 5 - protein, 9 aa, synthetic construct
ALWYSNLWV

SEQ ID NO: 6 - protein, 16 aa, synthetic construct
VRHGNFGNSYVSWFLY

SEQ ID NO: 7 - protein, 16 aa, synthetic construct
VRHGNFGNSYVSWFAY

SEQ ID NO: 8 - protein, 9 aa, synthetic construct
ALWYSNDWV

SEQ ID NO: 9 - protein, 5 aa, synthetic construct
ENIIH

SEQ ID NO: 10 - protein, 17 aa, synthetic construct
WFHPGSGSIKYNEKFKD

SEQ ID NO: 11 - protein, 11 aa, synthetic construct
HKGTGRGAMDY

SEQ ID NO: 12 - protein, 15 aa, synthetic construct
RASKSLSYGAYSYMH

SEQ ID NO: 13 - protein, 7 aa, synthetic construct
LASNLES

SEQ ID NO: 14 - protein, 9 aa, synthetic construct
QHSRELPYT

SEQ ID NO: 15 - protein, 485 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYQSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 485 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFLYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 485 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNDWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 485 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 450 aa, synthetic construct
QVQLVQSGAEVKKPGASVKVSCKASGYTFTENIIHWVRQAPGQGLEWIGWFHPGSGSIKYNEKFKDRVTMTADTSISTAYMELSRLRSDDTAVYYCARHKGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 218 aa, synthetic construct
DIVLTQSPASLAVSPGQRATITCRASKSLSYGAYSYMHWYQQKPGQPPKLLIYLASNLESGVPARFSGSGSGTDFTLTINPVEANDAANYYCQHSRELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 718 aa, synthetic construct
QVQLVQSGAEVKKPGASVKVSCKASGYTFTENIIHWVRQAPGQGLEWIGWFHPGSGSIKYNEKFKDRVTMTADTSISTAYMELSRLRSDDTAVYYCARHKGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 480 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 713 aa, synthetic construct
QVQLVQSGAEVKKPGASVKVSCKASGYTFTENIIHWVRQAPGQGLEWIGWFHPGSGSIKYNEKFKDRVTMTADTSISTAYMELSRLRSDDTAVYYCARHKGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 760 aa, synthetic construct
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 25 - DNA, 2280 bp, synthetic construct
  source 1..2280: mol_type = other DNA
atgaagacatgggtcaagatcgtattcggagtagctacctcagcagtgcttgccctgctcgtgatgtgcatcgttctgaggccatctcgagttcataatagcgaagaaaacacaatgcgtgctctcactctgaaggatattctcaacggtaccttttcttataagacattcttccctaactggatcagtggtcaggagtatttgcaccagtcagctgataacaacatagtcttgtacaacattgagactgggcagtcatacacaatcctgagtaataggactatgaaaagcgtcaatgctagtaattacggactgtccccagatcgccagttcgtgtatctggaaagtgactactctaagctctggagatactcttacaccgctacctattacatatacgatctgtctaacggcgagtttgtgagaggcaatgaacttcccagaccaattcaatacctgtgctggagcccagtaggaagcaaactggcttatgtgtatcagaacaatatctatctgaagcagaggcctggagatcctcctttccagataactttcaatgggcgggaaaacaagatctttaatggcattcccgactgggtctatgaggaagagatgctggccaccaagtacgctctgtggtggagccccaacggaaagtttctcgcctacgctgaattcaacgacaccgatataccagtgatcgcctatagttactatggtgatgagcagtatccaagaactattaatataccttatcccaaggctggagccaaaaacccagtggtacgaatttttatcatcgataccacctacccagcttacgtgggtccacaggaagtgccagttcccgccatgattgctagttcagactattacttttcttggctgacttgggtgaccgacgagcgcgtgtgccttcaatggcttaagcgcgtccaaaatgtgagcgtcctctcaatatgcgactttcgagaagactggcagacttgggattgccccaagacacaggagcatattgaagagagcagaactggttgggccggaggttttttcgtttctacacccgttttctcatacgacgccattagctactacaagatcttcagtgataaagacggctacaaacacatccactacatcaaggatactgttgaaaacgcaattcagatcactagcggaaagtgggaggccatcaacattttcagagtgacccaggacagcttgttctactcttcaaatgaatttgaggaatatcccggccgaaggaacatctatcggatctccatcggctcatatcctcccagtaaaaagtgcgtgacttgccatcttcgaaaggagagatgccagtactacactgcatcctttagcgactacgccaagtactacgcattggtgtgctacggacctggaattccaatctccaccctgcacgacggccgaaccgaccaggagatcaagatcctggaggagaacaaggagctggagaacgcactgaaaaacatccagctgccaaaagaagaaatcaagaaactggaagtggatgaaattaccctgtggtataagatgatcttgcctccacagtttgataggagcaagaaatacccactccttattcaagtctatgggggaccctgctctcagtctgtccggagcgtatttgccgtgaactggatttcttacctggcaagtaaggagggtatggtgattgctttggtggacggaagggggacagcatttcaaggggataagttgctgtatgcagtctacaggaagctgggagtctatgaagtggaagaccagattaccgccgttcgcaagttcatcgagatgggctttatcgacgaaaaacggattgctatttggggttggtcttacggcggttatgtgtctagcttggctttggcttccggcacaggtcttttcaagtgtggcatagctgtggcccctgtgagttcctgggaatattacgcctccgtatatacagagagatttatggggctgccaacaaaggatgacaacctcgagcactataagaattccacagttatggccagggcagaatactttcgtaacgtggactacctgttgatccatggcaccgctgatgataatgtccatttccagaactccgcacagatcgccaaggctctcgttaacgcccaggtggactttcaggctatgtggtactctgaccagaatcatgggctgagtggtttgtccactaatcacctgtacactcacatgacacatttcctgaagcagtgcttctccctgagcgac

SEQ ID NO: 26 - protein, 109 aa, synthetic construct
ELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 27 - protein, 125 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYQSWFAYWGQGTLVTVSS

SEQ ID NO: 28 - protein, 125 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFLYWGQGTLVTVSS

SEQ ID NO: 29 - protein, 109 aa, synthetic construct
ELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNDWVFGGGTKLTVL

SEQ ID NO: 30 - protein, 125 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 31 - protein, 111 aa, synthetic construct
DIVLTQSPASLAVSPGQRATITCRASKSLSYGAYSYMHWYQQKPGQPPKLLIYLASNLESGVPARFSGSGSGTDFTLTINPVEANDAANYYCQHSRELPYTFGGGTKLEIK

SEQ ID NO: 32 - protein, 120 aa, synthetic construct
QVQLVQSGAEVKKPGASVKVSCKASGYTFTENIIHWVRQAPGQGLEWIGWFHPGSGSIKYNEKFKDRVTMTADTSISTAYMELSRLRSDDTAVYYCARHKGTGRGAMDYWGQGTLVTVSS

SEQ ID NO: 33 - protein, 231 aa, synthetic construct
PKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 231 aa, synthetic construct
PKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 15 aa, synthetic construct
GGGGSGGGGSGGGGS

SEQ ID NO: 36 - protein, 5 aa, synthetic construct
GGGGS